(19) United States
(12) Patent Application Publication
(10) Pub. No.: US 2023/0116264 A1
(43) Pub. Date: Apr. 13, 2023

(54) COAGULATION ASSAYS FOR A POINT-OF-CARE PLATFORM

(71) Applicant: Baebies, Inc., Durham, NC (US)

(72) Inventors: Michael A. Basmajian, Durham, NC (US); Michael Boso, Durham, NC (US); Vijay SRINIVASAN, Durham, NC (US); Vamsee PAMULA, Durham, NC (US)

(73) Assignee: Baebies, Inc., Durham, NC (US)

(21) Appl. No.: 17/962,919

(22) Filed: Oct. 10, 2022

Related U.S. Application Data
(63) Continuation of application No. PCT/US2022/046071, filed on Oct. 7, 2022.
(60) Provisional application No. 63/253,810, filed on Oct. 8, 2021.
(60) Provisional application No. 63/350,259, filed on Jun. 8, 2022.

(51) Int. Cl. G01N 33/49; B01L 3/00; G16H 10/40

(52) U.S. Cl. CPC G01N 33/4905; B01L 3/502792; G16H 10/40; B01L 2400/0427; B01L 2300/087

(57) ABSTRACT

Coagulation assays for a point-of-care platform is disclosed. For example, the disclosure provides methods of measuring viscoelastic properties in a droplet on a microfluidics device, including using electrowetting-mediated droplet operations on the microfluidics device. In some embodiments, a microfluidics point-of-care platform may be used for assaying and/or monitoring coagulation of a blood sample. The disclosure provides a system, digital microfluidics device, and methods for measuring coagulation of a blood sample. In various embodiments, the disclosure provides a microfluidics device including droplets subject to manipulation by the device wherein droplet movement is used to characterize coagulation of a blood sample.

### CROSS-REFERENCE TO RELATED APPLICATIONS

This application is a 35 U.S.C. § 111(a) U.S. bypass continuation application of International Application No. PCT/US2022/046071 having an international filing date of Oct. 7, 2022, which claims priority to U.S. Patent App. No. 63/253,810, entitled “Coagulation Assay for Point-Of-Care Platform,” filed on Oct. 8, 2021; and U.S. Patent App. No. 63/350,259, entitled “Coagulation Assay for Point-Of-Care Platform,” filed on Jun. 8, 2022; the entire disclosures of which are incorporated herein by reference.

### GOVERNMENT RIGHTS TO THE DISCLOSURE

This disclosure was made with government support under Grant No. R44HL140662 and Grant No. R44HL125484 awarded by the National Institutes of Health (NIH). The government has certain rights in the disclosure.

### TECHNICAL FIELD

The subject matter relates generally to methods of measuring viscoelastic properties in a droplet using electrowetting-mediated droplet operations on a fluidics device and more particularly to monitoring coagulation of a blood sample using a microfluidics point-of-care platform.

### BACKGROUND

In clinical practice, monitoring blood coagulation and/or heparin levels in a blood sample is commonly used to target a therapeutic range of anticoagulation and as part of coagulation panels to help elucidate causes of bleeding or clotting disorders. For example, activated partial thromboplastin time (aPTT) and anti-Factor Xa are two tests that are commonly used to monitor and adjust dosage of heparin in patients requiring anticoagulation therapy. However, the current practice often requires frequent blood draws requiring relatively large volumes of sample, which may be problematic in certain patient populations, e.g., pediatric patients.

### SUMMARY

In one embodiment of the disclosure, a method is provided for measuring blood coagulation that includes: (a) on a microfluidics device, merging a blood sample droplet with an activation reagent that includes a coagulation-activator and with a coagulation initiation reagent to create a reaction droplet; (b) performing one or more shuttling impedance protocol cycles on the reaction droplet, wherein a single cycle comprises: transporting the reaction droplet from a first electrode across one or more electrodes to a destination electrode, wherein a measurement of impedance is taken at one or both the first and the destination electrodes early enough in a settling time of the reaction droplet for the measurement to be representative of droplet mobility; and (c) determining an ability of the blood sample droplet to form a clot and/or a time to clot formation when a change in the measured impedance meets or surpasses a predetermined threshold indicative of clot formation.

In one instance of the method, performing the single shuttling impedance protocol cycle includes: (a) holding the reaction droplet across the first electrode designated 1 and two electrodes designated 2 and 3 for about two seconds or more, wherein the reaction droplet is three reaction droplet units in size (3DU); (b) transporting the 3DU reaction droplet to electrodes 2-4 and holding for about 100 ms or less of settling time, wherein the destination electrode is designated 4; (c) taking a measurement of impedance at the first and the destination electrodes; (d) holding the 3DU reaction droplet across electrodes 2-4 for about two seconds or more; (e) transporting the 3DU reaction droplet to electrodes 1-3 and holding for about 100 ms or less of settling time; and (f) taking a measurement of impedance at the first and the destination electrodes.

In another embodiment, a method is provided for measuring blood coagulation that includes: (a) using electrowetting-mediated droplet operations on a microfluidics device: (i) dispensing a droplet of a blood sample in a droplet operations gap of the microfluidics device, (ii) initiating an “activated partial thromboplastin time” (aPTT) test by merging the sample droplet with an activation reagent droplet and an initiation reagent droplet to create a reaction droplet; and (iii) performing one or more shuttling impedance protocol cycles on the reaction droplet, a single cycle including transporting the reaction droplet from a first electrode to a destination electrode, wherein a measurement of impedance is taken at one or both of the first and destination electrodes early enough in a settling time of the reaction droplet for the measurement to be representative of droplet mobility; and (b) using a computer, determining an ability of the blood sample droplet to form a clot and/or a time to clot formation when a change in the measured impedance meets or surpasses a predetermined threshold indicative of clot formation.

In the methods described herein above, the activation reagent may be ellagic acid in a phospholipid solution or silica in a phospholipid solution, and the initiation reagent may overcome the effect of an anticoagulant agent used during initial collection of the blood sample. In one example, the initiation reagent comprises CaCl₂.

In some embodiments, the reaction droplet is held for a defined period of time after each measurement of impedance. The defined period of time may be about 1 second or more or about 2 seconds or more or about 3 seconds or more. In another embodiment defined period of time may be from about 1 second to about 10 seconds, or from about 1 second to about 5 seconds. In one instance, taking the impedance measurement early enough in a settling time of the reaction droplet for the measurement to be representative of droplet mobility is taking it at about 100 ms or less or about 50 ms or less.

In another example, a single cycle of the shuttling impedance protocol cycles includes transporting the reaction droplet from the first electrode to the destination electrode and transporting the reaction droplet back to the first electrode which becomes the destination electrode. In some instances, forty or fewer shuttling impedance protocol cycles are performed. In some instances, thirty or fewer shuttling impedance protocol cycles are performed. In some instances, ten or fewer shuttling impedance protocol cycles are performed.

In the methods of the disclosure for measuring blood coagulation, if the change in measured impedance is sudden it may be indicative of more severe clot clotting, and if the change in measured impedance is gradual it may be indicative of less severe clotting.

In another embodiment, a method is provided for measuring viscoelastic properties in a droplet that includes: (a) dispensing a droplet of a sample on a microfluidics device and, optionally, merging the droplet with one or more reaction reagents; (b) on the microfluidics device, performing one or more shuttling impedance protocol cycles on the droplet comprising transporting the droplet from a first electrode to a destination electrode, wherein a measurement of impedance is taken at one or both electrodes early enough in a settling time of the droplet for the measurement to be representative of droplet mobility; and (c) using a computer, determining a change in viscoelastic properties of the droplet by determining a change in the measured impedance. In the method, the reaction droplet may be transported from the first electrode across one or more electrodes to the destination electrode. In one instance, the one or more shuttling impedance protocol cycles comprises transporting the reaction droplet from the first electrode to the destination electrode and transporting the reaction droplet back to the first electrode which becomes the destination electrode.

In one aspect of the foregoing methods, the sample droplet is a blood sample droplet, and the blood sample droplet includes a test compound. In this aspect, an effect of the test compound on blood coagulation is determined by observing the change in measured impedance in the method relative to a reference blood sample droplet that does not include the test compound.

In another aspect of the foregoing methods, the sample droplet is a blood sample droplet, and the method further includes merging a test compound with the blood sample droplet. In this aspect, an effect of the test compound on blood coagulation is determined by observing the change in measured impedance in the method relative to a reference blood sample droplet that does not include the test compound.

In the methods of the disclosure, the measurement of impedance may be a measurement of current at the electrode for an applied voltage. In this instance, measured current is indicative of an amount of the reaction droplet present at the electrode where impedance is being measured. The measurement of impedance may be taken at each of the first and destination electrodes. The measurement of impedance may be taken each time the reaction droplet is transported.

In some embodiments, the microfluidics device includes one or more of a sample reservoir for loading and dispensing the sample, a diluent reservoir for dispensing a diluent solution, and one or more reagent reservoirs for dispensing one or more liquid assay reagents. In other embodiments, one or more of the reaction reagents is provided as a dried reagent spot on one or more droplet operations electrodes on the microfluidics device. The one or more dried reagent spots may be rehydrated using a diluent solution provided in a diluent reservoir.

In the methods of the disclosure, the blood sample or the sample may be, for example, a whole blood sample or a plasma sample. The plasma sample may be prepared on the microfluidics device from a whole blood sample by combining a whole blood sample droplet with an agglutination reagent and separating a plasma fraction from a red blood cell fraction using a plasma separation process.

In one aspect, the microfluidics device comprises an electrowetting cartridge and the dispensing, merging, transporting, combining, and/or initiating is performed using electrowetting-mediated droplet operations.

In the methods of the disclosure, the shuttling impedance protocol cycles may be performed in a reaction zone on the microfluidics device.

In another embodiment, a system is provided that includes a computer processor and an electrowetting cartridge. The computer processor is programmed to execute any one of the methods disclosed herein.

In another aspect, a kit is provided that includes an electrowetting cartridge and reagents sufficient to execute any one of the methods disclosed herein.

In one aspect, the present disclosure is directed to a method for measuring blood coagulation, the method comprising: (a) on microfluidics device, merging a blood sample droplet with an activation reagent comprising a coagulation-activator and with a coagulation initiation reagent to create a reaction droplet; (b) performing one or more shuttling impedance protocol cycles on the reaction droplet, wherein a single cycle of the one or more shuttling impedance protocol cycles comprises: transporting the reaction droplet from a first electrode across one or more electrodes to a destination electrode, wherein a measurement of impedance is taken at one or both of the first electrode and/or the destination electrode; and (c) determining an ability of the blood sample droplet to form a clot when a change in the measured impedance meets or surpasses a predetermined threshold indicative of clot formation.

In some embodiments, performing the single shuttling impedance protocol cycle comprises: (a) holding the reaction droplet across the first electrode designated 1 and two electrodes designated 2 and 3 for about two seconds or more, wherein the reaction droplet is three reaction droplet units in size (3DU); (b) transporting the 3DU reaction droplet to electrodes 2-4 and holding for about 100 ms or less of settling time, wherein electrode 4 is the destination electrode; (c) taking a measurement of impedance at the first electrode and the destination electrode; (d) holding the 3DU reaction droplet across electrodes 2-4 for about two seconds or more; (e) transporting the 3DU reaction droplet to electrodes 1-3 and holding for about 100 ms or less of settling time; and (f) taking a measurement of impedance at the first electrode and the destination electrode.

In another aspect the present disclosure is directed to a method for measuring blood coagulation, comprising: (a) using electrowetting-mediated droplet operations on a microfluidics device: (i) dispensing a droplet of a blood sample in a droplet operations gap of the microfluidics device, (ii) initiating an activated partial thromboplastin time (aPTT) test by merging the sample droplet with an activation reagent droplet and an initiation reagent droplet to create a reaction droplet; and (iii) performing one or more shuttling impedance protocol cycles on the reaction droplet, wherein a single cycle of the one or more shuttling impedance protocol cycles comprises transporting the reaction droplet from a first electrode to a destination electrode, wherein a measurement of impedance is taken at one or both of the first electrode and/or the destination electrode; and (b) using a computer, determining an ability of the blood sample droplet to form a clot when a change in the measured impedance meets or surpasses a predetermined threshold indicative of clot formation.

In some embodiments, the method further comprising measuring the change in impedance over time and determining a time to clot formation when a change in the measured impedance meets or surpasses a predetermined threshold indicative of clot formation.

In some embodiments, the activation reagent comprises ellagic acid in a phospholipid solution or silica in a phospholipid solution.

In some embodiments, the initiation reagent overcomes the effect of an anticoagulant agent used during initial collection of the blood sample. In some embodiments, the initiation reagent comprises CaCl₂.

In some embodiments, the reaction droplet is held for a defined period of time after each measurement of impedance. In some embodiments the defined period of time comprises about 2 seconds or more.

In some embodiments, the single cycle comprises transporting the reaction droplet from the first electrode across the one or more electrodes to the destination electrode and transporting the reaction droplet back to the first electrode which becomes the destination electrode.

In some embodiments, forty or fewer shuttling impedance protocol cycles are performed.

In some embodiments, if the change in measured impedance is sudden it is indicative of more severe clot clotting, and wherein if the change in measured impedance is gradual it is indicative of less severe clotting.

In some embodiments, the measurement of impedance is a measurement of current at the electrode for an applied voltage.

In another aspect the present disclosure is directed to a method for measuring viscoelastic properties in a droplet, the method comprising: (a) dispensing a droplet of a sample on a microfluidics device and, optionally, merging the droplet with one or more reaction reagents; (b) on the microfluidics device, performing one or more shuttling impedance protocol cycles on the droplet, wherein the one or more shuttling impedance protocol cycles comprises transporting the droplet from a first electrode to a destination electrode, wherein a measurement of impedance is taken at one or both of the electrodes; and (c) using a computer, determining a change in viscoelastic properties of the droplet by determining a change in the measured impedance.

In some embodiments, the reaction droplet is transported from the first electrode across one or more electrodes to the destination electrode.

In some embodiments, the one or more shuttling impedance protocol cycles comprises transporting the reaction droplet from the first electrode to the destination electrode and transporting the reaction droplet back to the first electrode which becomes the destination electrode.

In some embodiments, the measurement of impedance is a measurement of current at the electrode for an applied voltage. In some embodiments, the measured current is indicative of an amount of the reaction droplet present at the electrode.

In some embodiments, the measurement of impedance is taken early enough in a settling time of the reaction droplet for the measurement to be representative of droplet mobility, and wherein early enough in the settling time comprises about 100 ms or less.

In some embodiments, the measurement of impedance is taken at each of the first and the destination electrodes. In other embodiments, the measurement of impedance is taken each time the reaction droplet is transported.

In some embodiments, the sample droplet is a blood sample droplet comprising a test compound, wherein an effect of the test compound on blood coagulation is determined by the change in measured impedance relative to a reference blood sample droplet that does not comprise the test compound.

In some embodiments, the method further comprising merging the blood sample droplet with a test compound, wherein an effect of the test compound on blood coagulation is determined by the change in measured impedance relative to a reference blood sample droplet that does not comprise the test compound.

In some embodiments, the microfluidics device comprises one or more of a sample reservoir for loading and dispensing the sample, a diluent reservoir for dispensing a diluent solution, and one or more reagent reservoirs for dispensing one or more liquid assay reagents.

In some embodiments, one or more of the reaction reagents is provided as a dried reagent spot on one or more droplet operations electrodes on the microfluidics device. In some embodiments, the one or more dried reagent spots are rehydrated using a diluent solution provided in a diluent reservoir.

In some embodiments, the microfluidics device comprises an electrowetting cartridge and the dispensing, merging, transporting, combining, and/or initiating is performed using electrowetting-mediated droplet operations.

In some embodiments, the shuttling impedance protocol cycles are performed in a reaction zone on the microfluidics device.

In some embodiments, the blood sample or the sample is a whole blood sample or a plasma sample.

In some embodiments, the plasma sample is prepared on the microfluidics device from a whole blood sample by combining a whole blood sample droplet with an agglutination reagent and separating a plasma fraction from a red blood cell fraction using a plasma separation process.

In another aspect, the present disclosure is directed to a system comprising a computer processor and an electrowetting cartridge wherein the processor is programmed to execute the any of the methods described herein.

In still another aspect, the present disclosure is directed to a kit comprising an electrowetting cartridge and reagents sufficient to execute any of the methods described herein.

### BRIEF DESCRIPTION OF DRAWINGS

Having thus described the subject matter in general terms, reference will now be made to the accompanying drawings, which are not necessarily drawn to scale, and wherein:

FIG. 1 illustrates a simplified block diagram of an example of a microfluidics system for performing blood coagulation assays in droplets;

FIG. 2 illustrates a cross-sectional view of an example of a portion of a microfluidics device for performing a blood coagulation assay in droplets;

FIG. 3A and FIG. 3B illustrate schematic diagrams of an example of an arrangement of droplet operations electrodes configured for conducting an aPTT assay for blood clotting on a microfluidics device using liquid reagents;

FIG. 4A, FIG. 4B, and FIG. 4C illustrate schematic diagrams of an example of a droplet shuttling impedance protocol for measuring time to clot formation;

FIG. 5 illustrates a plot showing an example of detecting droplet mobility using impedance measurements.

FIG. 6 illustrates a flow diagram of an example of a method of measuring clotting of a blood sample on a microfluidics device using an aPTT assay;

FIG. 7 illustrates a plot showing impedance traces for clot formation in whole blood reaction droplets;

FIG. 8 illustrates a plot showing impedance traces for clot formation in a blood sample at different settling times in a droplet shuttling impedance protocol;

FIG. 9A and FIG. 9B illustrate cross-sectional views of a portion of a microfluidics device and showing an example of a droplet shuttling impedance protocol for use in the blood coagulation assays of the disclosure;

FIG. 10A and FIG. 10B illustrate cross-sectional views of a portion of microfluidics device 110 and showing another example of a droplet shuttling impedance protocol for use in the blood coagulation assays of the disclosure;

FIG. 11A, FIG. 11B, and FIG. 11C illustrate plan views showing an example of an electrode configuration including flanking electrodes and showing another example of a droplet shuttling impedance protocol for use in the blood coagulation assays of the disclosure;

FIG. 12A, FIG. 12B, and FIG. 12C illustrate plan views showing an example of an electrode configuration including flanking electrodes and showing another example of a droplet shuttling impedance protocol for use in the blood coagulation assays of the disclosure;

FIG. 13A, FIG. 13B, FIG. 13C, and FIG. 13D illustrate plan views showing an example of a circular electrode configuration and showing another example of a droplet shuttling impedance protocol for use in the blood coagulation assays of the disclosure;

FIG. 14 illustrates a plot showing impedance traces for example clot formation of a droplet shuttling impedance protocol; and

FIG. 15 is a cross-sectional view of a portion of a microfluidics device and showing an example of a droplet shuttling optical imaging protocol for use in the blood coagulation assays of the disclosure.

### DETAILED DESCRIPTION

The disclosure provides coagulation assays for a point-of-care platform. For example, the subject matter includes monitoring coagulation of a blood sample using a microfluidics point-of-care platform.

In one embodiment, the disclosure provides a system, digital microfluidics device, and methods for measuring viscoelastic properties in a droplet. The methods include: (a) dispensing a droplet of a sample on a microfluidics device and, optionally, merging the droplet with one or more reaction reagents; (b) on the microfluidics device, performing one or more shuttling impedance protocol cycles on the droplet comprising transporting the droplet from a first electrode to a destination electrode, wherein a measurement of impedance is taken at one or both electrodes; and (c) using a computer, determining a change in viscoelastic properties of the droplet by determining a change in the measured impedance. Optionally, the measurement of impedance can be taken early enough in a settling time of the droplet for the measurement to be representative of droplet mobility, e.g., within about 100 ms or less or within about 50 ms or less.

In the methods, the reaction droplet may be transported from the first electrode across one or more electrodes to the destination electrode. In some instances, the one or more shuttling impedance protocol cycles comprises transporting the reaction droplet from the first electrode to the destination electrode and transporting the reaction droplet back to the first electrode which then becomes the destination electrode. In other instances, the droplet may be transported on a long path (for example, a loop), with impedance measured at one or both of each new destination/trailing electrode, or at any set of electrodes along the path.

In another embodiment, the disclosure provides a system, digital microfluidics device, and methods for measuring viscoelastic properties in a droplet. The methods include: (a) dispensing a droplet of a sample on a microfluidics device and, optionally, merging the droplet with one or more reaction reagents; (b) on the microfluidics device, performing one or more shuttling optical imaging protocol cycles on the droplet comprising transporting the droplet from a first electrode to a destination electrode, wherein a digital image is captured at one or both electrodes using a camera (e.g., RGB color camera); and (c) using a computer with image processing capabilities, determining a change in viscoelastic properties of the droplet to determine, for example, a change in the size, shape, color, and/or position of the droplet. Optionally, the digital image may be captured early enough in a settling time of the droplet for the contents of the digital image to be representative of droplet mobility, e.g., within 100 ms or less or within 10 ms or less.

In the methods, the reaction droplet may be transported from the first electrode across one or more electrodes to the destination electrode. In some instances, the one or more shuttling optical imaging protocol cycles comprises transporting the reaction droplet from the first electrode to the destination electrode and transporting the reaction droplet back to the first electrode which then becomes the destination electrode. In other instances, the droplet may be transported on a long path (for example, a loop), with images captured at one or both of each new destination/trailing electrode or at any of the electrodes along the path.

The system provided may include a computer processor and an electrowetting cartridge. The computer processor can be programmed to execute any one of the methods disclosed herein.

In another aspect, a kit is provided that includes an electrowetting cartridge and reagents sufficient to execute any one of the methods disclosed herein.

### Coagulation Assay for Point-of-Care Platform

In some embodiments, the disclosure provides a system, digital microfluidics device, and methods for measuring coagulation of a blood sample.

In various embodiments, the disclosure provides a microfluidics device including droplets subject to manipulation by the device wherein droplet movement is used to characterize coagulation of a blood sample.

In one embodiment, a blood sample may be evaluated for the ability to form a blood clot.

In one embodiment, a blood sample may be evaluated for the time required to form a blood clot.

In some embodiments, the methods of the disclosure make use of two consecutive series of biochemical reactions to monitor the formation of a clot and/or the time-to-formation (i.e., speed) of a clot in a blood sample droplet.

In some embodiments, the time to clot formation may be monitored using a mechanical detection process. For example, a velocity-based detection process using repeated shuttling of a sample droplet may be used to determine time to clot formation of the blood sample.

In another embodiment, the velocity-based detection process uses impedance measurements as a coagulation assay readout to determine the presence or absence of a clot and/or the speed of clot formation in a blood sample droplet.

In one instance, the sample droplet is a blood sample droplet, and the blood sample droplet includes a test compound. In this instance, an effect of the test compound on blood coagulation is determined by observing the change in measured impedance in the method relative to a reference blood sample droplet that does not include the test compound.

In another instance, the sample droplet is a blood sample droplet, and the method further includes merging a test compound with the blood sample droplet. In this instance, an effect of the test compound on blood coagulation is determined by observing the change in measured impedance in the method relative to a reference blood sample droplet that does not include the test compound.

### Microfluidic System, Device, and Methods

Fluidics systems and devices are used in a variety of applications to manipulate, process and/or analyze biological materials. Examples of fluidics devices include droplet actuators, microfluidics cartridges, digital microfluidics (DMF) devices, DMF cartridges, flow cell devices, and the like. The disclosure provides a microfluidic system, device, and methods including droplets subject to manipulation by the device wherein droplet movement is used to characterize coagulation of a blood sample.

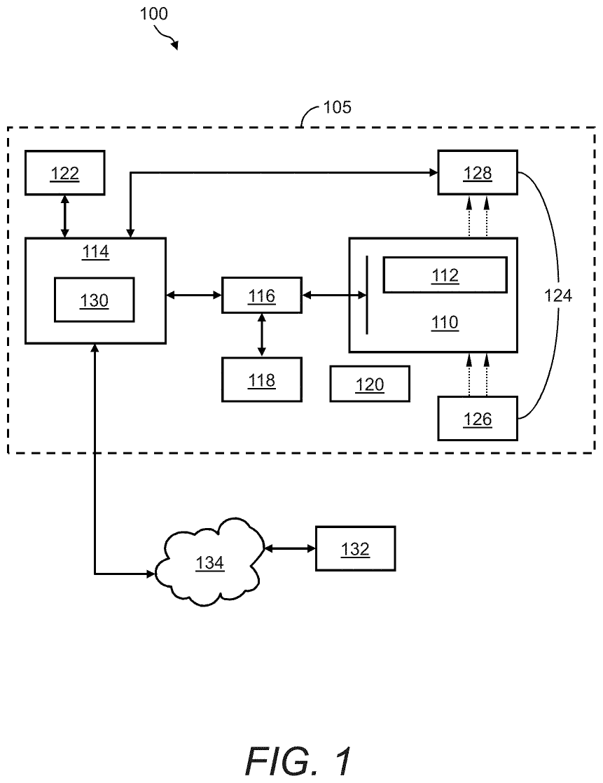

Referring now to FIG. 1 is a simplified block diagram of an example of a microfluidics system 100 for performing blood coagulation assays in droplets. In this example, microfluidics system 100 may include a fluidics instrument 105. Further, fluidics instrument 105 may house a microfluidics cartridge 110 along with any supporting components. Microfluidics cartridge 110 of microfluidics system 100 may be, for example, any fluidics device or cartridge, microfluidics device or cartridge, DMF device or cartridge, droplet actuator, flow cell device or cartridge, and the like. In various embodiments, microfluidics system 100 provides microfluidics cartridge 110 that may support automated processes to manipulate, process, and/or analyze biological materials.

Microfluidics cartridge 110 may be provided, for example, as a disposable and/or reusable device or cartridge. Microfluidics cartridge 110 may be used for processing biological materials. For example, blood coagulation assays 112 may be executed on microfluidics cartridge 110 using, for example, droplet operations.

Also housed in fluidics instrument 105 of microfluidics system 100 may be a controller 114, a cartridge interface 116, certain thermal control electronics 118, one or more magnets 120, a graphical user interface (GUI) 122, and a detection system 124. Controller 114 may be electrically coupled to the various hardware components of fluidics instrument 105. For example, controller 114 may be electrically coupled to microfluidics cartridge 110, thermal control electronics 118, magnets 120, GUI 122, and detection system 124. In particular, controller 114 may be electrically coupled to microfluidics cartridge 110 via cartridge interface 116, wherein cartridge interface 116 may be, for example, a pluggable interface for connecting mechanically and electrically to microfluidics cartridge 110.

Most chemical and biological processes have precise and stable temperature control for optimal efficiency and performance Accordingly, thermal control electronics 118 may be any mechanisms for controlling the operating temperature of microfluidics cartridge 110. Thermal control electronics 118 may include, for example, any thermal sensors for controlling heaters (e.g., Peltier elements and resistive heaters). Thermal control electronics 118 may also include coolers arranged with respect to microfluidics cartridge 110.

Magnets 120 may be, for example, permanent magnets and/or electromagnets. In the case of electromagnets, controller 114 may be used to control the electromagnets 120. GUI 122 may be any type of digital display for conveying information to a user. In one example, GUI 122 may be the display of fluidics instrument 105. In another example, GUI 122 may be the display of any networked computing device connected to fluidics instrument 105 via a network. For example, a networked computer 132 maybe connected to fluidics instrument 105 via a network 134.

Networked computer 132 may be, for example, any centralized server or cloud-based server. Network 134 may be, for example, a local area network (LAN) or wide area network (WAN) for connecting to the internet. The communications interface (not shown) of controller 114 may be any wired and/or wireless communication interface for connecting to a network (e.g., network 134). Information may be exchanged with other devices connected to the network. Though FIG. 1 shows a single networked computer 132, multiple computers (physical or virtual) may be connected in the microfluidics system 100 via network 134. For example, one computer may be used to control thermal control electronics 118. Then, another computer may be used to control electromagnets 120. Then, another computer that is optimized for storing information may be used to store data received from detection system 124. Then, yet another computer that is optimized for processing data may be used to process information received from fluidics instrument 105.

Controller 114 may, for example, be a general-purpose computer, special-purpose computer, personal computer, tablet device, smart phone, smart watch, mobile device, microprocessor, or other programmable data processing apparatus. Controller 114 may provide processing capabilities, such as storing, interpreting, and/or executing software instructions. Additionally, controller 114 may be used to control the overall operations of microfluidics system 100. The software instructions may comprise machine-readable code stored in non-transitory memory that is accessible by controller 114 for the execution of the instructions. Controller 114 may be configured and programmed to control data and/or power aspects of microfluidics system 100. Further, data storage (not shown) may be built into or provided separate from controller 114.

Further, in some embodiments, controller 114 may be external to fluidics instrument 105 (not shown in FIG. 1). The functions described above may be done remotely, for example via a mobile application running on a mobile device connected to various components (i.e., thermal controls electronics 118, among others) via a local network or other network. Output from detection system 124 may also be transmitted to an external controller 114 via such networks and displayed on a mobile application or another mobile app running on a mobile device specific for the external controller 114.

Generally, controller 114 may be used to manage any functions of microfluidics system 100. For example, controller 114 may be used to manage the operations of thermal control electronics 118, magnets 120, GUI 122, detection system 124, and any other instrumentation (not shown) in relation to microfluidics cartridge 110. Further, with respect to microfluidics cartridge 110, controller 114 may control droplet manipulation by activating/deactivating electrodes.

In other embodiments of microfluidics system 100, the functions of controller 114, thermal control electronics 118, magnets 120, GUI 122, detection system 124, and/or any other instrumentation may be integrated directly into microfluidics cartridge 110 rather than provided separately from microfluidics cartridge 110.

Detection system 124 may be, for example, an optical measurement system that includes an illumination source 126 and an optical measurement device 128. In this example, illumination source 126 and optical measurement device 128 may be arranged with respect to microfluidics cartridge 110.

In one example, detection system 124 may be provided in relation to the certain detection spots corresponding to blood coagulation assays 112 running on microfluidics cartridge 110. With respect to performing blood coagulation assays in droplets, detection system 124 (e.g., the camera) may be used to confirm the presence, amount or absence of a droplet at a specific electrode position. For example, the presence of a droplet at a destination electrode following a droplet operations event. Thereby, confirming that the droplet operations event was effective. Additionally, detection system 124 may be any detection mechanism that can be used to sensitively detect any biological materials of interest present in a sample. Detection system 124 may include, for example, one or more excitation light sources and one or more optical detectors or sensors of any types.

Illumination source 126 of detection system 124 may be, for example, a light source for the visible range (400-800 nm), such as, but not limited to, a white light-emitting diode (LED), a halogen bulb, an arc lamp, an incandescent lamp, lasers, and the like. Illumination source 126 is not limited to a white light source. Illumination source 126 may be any color light that is useful in microfluidics system 100. Optical measurement device 128 may be used to obtain light intensity readings. Optical measurement device 128 may be, for example, a charge coupled device, a photodetector, a spectrometer, a photodiode array, a digital camera (e.g., RGB color camera) or any combinations thereof. Further, microfluidics system 100 is not limited to one detection system 124 only (e.g., one illumination source 126 and one optical measurement device 128 only). Microfluidics system 100 may include multiple detection systems 124 (e.g., multiple illumination sources 126 and/or multiple optical measurement devices 128).

Additionally, in some embodiments, microfluidics cartridge 110 may include certain feedback mechanisms, such as impedance and/or capacitance sensing or imaging techniques, which may be used to determine or confirm the outcome of a droplet operation. For example, controller 114 may include impedance sensing 130. In one example, sensors may be embedded at each droplet operations electrode location of microfluidics cartridge 110 to measure impedance. These sensors may be driven and/or controlled by circuitry of impedance sensing 130. Impedance sensing 130 enables monitoring and closed-loop control of certain droplet operations.

With respect to performing blood coagulation assays in droplets, impedance sensing 130 may be used to confirm the presence, amount or absence of a droplet at a specific electrode position. For example, the presence of a droplet at a destination electrode following a droplet operations event. Thereby, confirming that the droplet operations event was effective. Additionally, impedance sensing 130 that may be used for analyzing blood coagulation assays in droplets (e.g., blood coagulation assays 112).

Likewise, optical measurement device 128 (e.g., camera) of detection system 124 may be used to confirm the presence, amount or absence of a droplet at a specific electrode position. Accordingly, optical measurement device 128 that may be used for analyzing blood coagulation assays in droplets (e.g., blood coagulation assays 112). Further, impedance sensing 130 and optical measurement device 128 (e.g., camera) of detection system 124 may be used together or separately for analyzing blood coagulation assays.

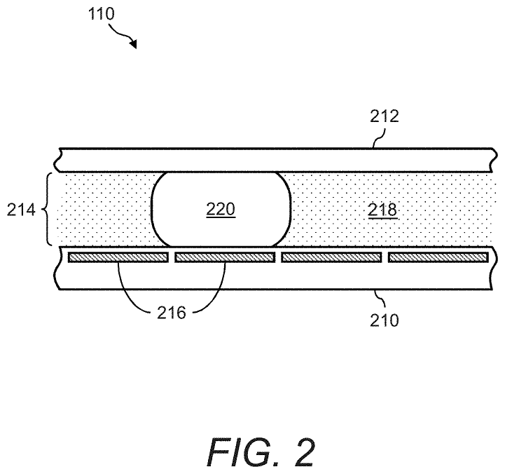

Referring now to FIG. 2 is a cross-sectional view of an example of a portion of microfluidics cartridge 110 for performing a blood coagulation assay in droplets. Microfluidics cartridge 110 may include a bottom substrate 210 and a top substrate 212 separated by a droplet operations gap 214. In some embodiments top substrate 212 may be absent, and the methods of the disclosure may be carried out on a bottom substrate 210. A set of droplet operations electrodes 216 (e.g., electrowetting electrodes) may be arranged, for example, on bottom substrate 210. The droplet operations electrodes 216 are arranged for conducting droplet operations, such as droplet loading, dispensing, splitting, transporting, merging, and mixing. Droplet operations gap 214 may be filled with a filler fluid 218. Filler fluid 218 may, for example, be a low-viscosity oil, such as silicone oil. In some cases, filler fluid 218 is absent, and the methods of the disclosure are carried out in air or in a gas environment. In some cases, both filler fluid 218 and top substrate 212 absent while conducting the methods of the disclosure.

An aqueous droplet 220 may be present in droplet operations gap 214 of microfluidics cartridge 110. In one example, droplet 220 may be a droplet of a blood sample to be evaluated, such as a whole blood droplet or a plasma droplet. In another example, droplet 220 may be a reagent droplet for conducting a coagulation assay, such as first reagent droplet, or a second reagent droplet. Oil filler fluid 218 may fill droplet operations gap 214 and surround droplet 220.

### Coagulation (or “Clot-Based”) Assays

The disclosure provides methods for evaluating coagulation (i.e., clotting) of a blood sample on a microfluidics device. In various embodiments, the methods may be used to assess changes in the viscoelastic properties of a blood sample droplet in a microfluidics device, wherein the droplet is subject to manipulation by the device and droplet movement is used to characterize clotting in the blood sample.

The methods of the invention make use of biochemical reactions in the coagulation cascade to assess formation of a clot in a blood sample droplet in a microfluidics device, wherein the droplet is subject to manipulation by the device and droplet movement is used to characterize clotting of the blood sample.

In some embodiments, the methods may be used to assess the endpoint in clot formation.

In some embodiments, the methods may be used to assess the time-to-formation of a clot in a blood sample.

In some embodiments, the methods may be used to assess the rate of clot formation in a blood sample.

In some embodiments, the blood sample is a whole blood sample. A whole blood sample may, for example, be collected using an appropriate anticoagulant agent (e.g., sodium citrate) to inhibit coagulation of the blood sample prior to testing.

In some embodiments, the blood sample is a plasma sample. The plasma sample may, for example, be prepared from a whole blood sample collected using an appropriate anticoagulant (e.g., sodium citrate) to inhibit coagulation. A plasma sample for use in the methods of the invention may, for example, be prepared on the microfluidics device from a whole blood sample by combining a whole blood sample droplet with an agglutination reagent and separating a plasma fraction from a red blood cell fraction using a plasma separation process. In one example, the agglutination reagent may be a hemagglutinating lectin (e.g., Solanum Tuberosum Lectin “SGL” or “potato lectin”).

Several clot-based coagulation assays that target one or more pathways in the coagulation cascade are known in the art. For example, a clot-based assay may be used to assess the extrinsic, intrinsic, and/or common pathways in the coagulation cascade that lead to formation of a clot (see, e.g., Cornell University College of Veterinary Medicine eclinpath.com/hemostasis/tests/screening-coagulation-assays/; Gates, S. M., and Weitz, J. I. (2005) Circulation 112: e53-e60; Seasor, T., and Moser, K. A., PathologyOutlines.com https://www.pathologyoutlines.com/topic/coagulationmixingstudies.html.; and Stang, L. J., and Mitchell, L. G. (2013) Methods Mol Biol. 992: 181-92, which are incorporated herein by reference in their entireties). The extrinsic and intrinsic coagulation pathways are activated in response to external trauma and internal damage, respectively, to the vascular endothelium and converge at a point in the common pathway to continue formation of the clot (i.e., the common pathway activates fibrinogen to fibrin to form the clot). Several clotting factors in each pathway are involved in the coagulation cascade. For example, clotting factors involved in the extrinsic pathway include, for example, factors VII and III; clotting factors involved in the intrinsic pathway include, for example, factors XII, XI, IX, and VIII; and clotting factors involved in the common pathway include, for example, factors X, V, II, I, and XIII

A conventional clot-based assay may be modified to assess formation of a clot in a blood sample droplet in a microfluidics device. For example, a clot-based assay may be modified for use on a microfluidics device, wherein the droplet is subject to manipulation by the device and droplet movement is used to characterize clotting of the blood sample. In various embodiments, a clot-based assay may be modified for use on a microfluidics device to assess the extrinsic, intrinsic, and/or common pathways in the coagulation cascade leading to formation of a clot.

In various embodiments, a clot-based assay of the invention generally includes: (i) an activation step that includes combining a blood sample droplet (e.g., a plasma droplet or a whole blood sample droplet) with a coagulation-activator, wherein the coagulation-activator may be provided in a phospholipid solution; (ii) an optional initiation step to start the coagulation event (i.e., clotting), wherein an initiation reagent is used to overcome the effect of an anticoagulant agent that may be used during the initial collection of the blood sample; and (iii) a detection step to determine the presence or absence of clot formation and/or the time-to-clot formation (i.e., the conversion of fibrinogen to fibrin or the “speed of clot formation”). Examples of activation reagents include, but are not limited to, tissue factor (e.g., tissue thromboplastin or recombinant tissue factor); solutions of ellagic acid or silica in a phospholipid solution; and snake venom (e.g., ecarin). Examples of an initiation reagent include calcium solutions (e.g., CaCl₂).

In various embodiments, the methods may be used to assess states of “aberrant” coagulation that may be linked to the extrinsic, intrinsic, and/or common pathways of coagulation.

In some embodiments, the methods make use of biochemical reactions in the extrinsic and common pathways of coagulation to assess formation of a clot and/or the time-to-formation of a clot in a blood sample droplet in a microfluidics device, wherein the droplet is subject to manipulation by the device and droplet movement is used to characterize clotting of the blood sample.

In one embodiment, a modified “prothrombin time” (PT) test may be used to assess the formation of a blood clot and/or the speed of clot formation in a blood sample droplet. In one example, a modified PT assay may include: (i) an initiation step that includes combining a blood sample droplet with a thromboplastin reagent that includes a calcium reagent to overcome the effect of an anticoagulant agent used during collection of the blood sample; and (ii) a detection step to determine the time-to-clot formation (i.e., speed of clot formation). In one example, the thromboplastin reagent may include a mixture of tissue factor, phospholipid, and calcium.

In another embodiment, a modified “protein induced by vitamin K absence” (PIVKA) test may be used to assess the formation of a blood clot and/or the speed of clot formation in a blood sample droplet. In one example, a modified PIVKA assay may include: (i) an initiation step that includes combining a blood sample droplet (e.g., a diluted plasma sample droplet) with an initiating reagent that includes calcium to overcome the effect of an anticoagulant agent used during collection of the blood sample; and (ii) a detection step to determine the time-to-clot formation (i.e., speed of clot formation). In some cases, a commercially prepared tissue thromboplastin reagent, which contains tissue factor, a source of phospholipid, and calcium may be used to activate and initiate the clotting reaction in a single step.

In some embodiments, the methods make use of biochemical reactions in the intrinsic and common pathways of coagulation to assess the formation of a clot and/or the time-to-formation of a clot in a blood sample droplet in a microfluidics device, wherein the droplet is subject to manipulation by the device and droplet movement is used to characterize clotting of the blood sample.

In one embodiment, a modified “activated partial thromboplastin time” (aPTT) test may be used to assess the formation of a blood clot and/or the speed of clot formation in a blood sample droplet. In one example, a modified aPTT test may include: (i) an activation step that includes combining a blood sample droplet (i.e., a plasma droplet) with a coagulation-activator (e.g., kaolin, celite, ellagic acid, or silica) in a phospholipid solution; (ii) an initiation step to start the coagulation event (i.e., clotting), wherein calcium reagent (e.g., CaCl₂) is used to overcome the effect of an anticoagulant agent used during the initial collection of the blood sample; and (iii) a detection step to determine the time-to-clot formation (i.e., speed of clot formation). In some cases, after combining the plasma droplet with the coagulation-activator, an incubation step (e.g., about 3 minutes) is performed to allow activation of contact factors (e.g., factor XII, factor XI, and others) prior to addition of the calcium reagent.

In another embodiment, a modified “activated coagulation time” (ACT) test may be used to assess the formation of a blood clot and/or the speed of clot formation in a blood sample droplet. In one example, a modified ACT test may include: (i) an activation step that includes combining a whole blood sample droplet with a coagulation-activator (e.g., an activator of the intrinsic pathway such as kaolin, celite, or glass particles); and (ii) a detection step to determine the time-to-clot formation (i.e., speed of clot formation).

In some embodiments, the methods make use of biochemical reactions in the common pathway of coagulation to assess the formation of a clot and/or the time-to-formation of a clot in a blood sample droplet in a microfluidics device, wherein the droplet is subject to manipulation by the device and droplet movement is used to characterize clotting of the blood sample. For example, an assay may be performed to assess the amount and/or function of fibrinogen in a sample by combining a blood sample droplet with a thrombin reagent, wherein thrombin mediates conversion of fibrinogen to fibrin for clot formation.

In one embodiment, a modified “thrombin clot time” (TCT) test may be used to assess the amount or function of fibrinogen in a blood sample droplet. In one example, a TCT assay may include: (i) an activation step that includes combining a blood sample droplet (i.e., a plasma droplet) with an excess of a thrombin reagent; and (ii) a detection step to determine the time-to-clot formation (i.e., speed of clot formation).

In another embodiment, a modified “Clauss fibrinogen” assay may be used to assess the formation of a blood clot and/or the speed of clot formation in a blood sample droplet. In one example, a fibrinogen assay may include: (i) an activation step that includes combining a diluted plasma droplet with an excess of a coagulation-activator, i.e., thrombin; (ii) an initiation step to start the coagulation event (i.e., clotting), wherein a calcium reagent is used to overcome the effect of an anticoagulant agent used during the initial collection of the blood sample; and (iii) a detection step to determine the time-to-clot formation (i.e., speed of clot formation).

In various embodiments, the methods may be used to monitor and assess the effect of an anticoagulation drug therapy (e.g., a thrombin inhibitor) on clot formation. For example, a blood sample droplet in a microfluidics device may be combined with an anticoagulant reagent droplet to yield a combined sample/reagent droplet, wherein the combined sample/reagent droplet is subject to manipulation by the device and droplet movement is used to characterize clotting of the blood sample.

In one embodiment, a modified “ecarin clotting time” (ECT) test may be used to monitor and assess the effect of a thrombin inhibitor (e.g., hirudin) on the formation of a blood clot and/or the speed of clot formation in a blood sample droplet. Generally, in an ECT test an “activator” (i.e., ecarin derived from snake venom) is used to convert prothrombin to the active intermediate meizothrombin that is sensitive to inhibition by direct thrombin inhibitors. Inhibition of meizothrombin activity by the thrombin inhibitor may then be assessed. In one example, a modified ECT test may include: (i) an activation step wherein a blood sample droplet (i.e., a diluted plasma droplet) is combined with an activator ecarin reagent droplet; and (ii) a detection step to determine the time-to-clot formation (i.e., speed of clot formation).

In various embodiments, the methods may be used as a screening and/or monitoring tool for assessing the production and/or activity of autoimmune proteins (e.g., antiphospholipid antibodies or lupus anticoagulants) that may cause aberrant blood clotting. For example, a blood sample droplet in a microfluidics device may be combined with an activation reagent droplet (e.g., a clotting factor X activator) to yield a combined sample/reagent droplet, wherein the combined sample/reagent droplet is subject to manipulation by the device and droplet movement is used to characterize clotting of the blood sample.

In one embodiment, a modified “dilute Russell viper venom test/time” (dRVVT) test may be used as a screening and/or monitoring tool for assessing the production and/or activity of autoimmune proteins (e.g., anti-phospholipid antibodies or lupus anticoagulants). The dRVVT test uses “Russell's viper venom” (from a Daboia species) as an activating agent. In one example, a modified dRVVT test may include: (i) an activation step wherein a blood sample droplet (i.e., plasma droplet) is combined with an activator reagent droplet comprising diluted Russell's viper venom and a phospholipid solution; and (ii) an initiation step to start the coagulation event (i.e., clotting), wherein a calcium reagent is used to overcome the effect of an anticoagulant agent used during the initial collection of the blood sample; and (iii) a detection step to determine the time-to-clot formation (i.e., speed of clot formation).

In some embodiments, a “mixing study” may be performed to investigate abnormal clotting time results identified in a coagulation assay (e.g., a prolonged clotting time). For example, a mixing study may be used to aid in determining whether a prolonged clotting time representative of a clotting factor deficiency or an inhibitor. Generally, in a mixing study, a patient plasma sample is combined at different ratios with a normal pooled plasma sample (e.g., patient: pooled plasma ratio of 1:1, 4:1) and the clotting test is performed. For example, if the abnormal clotting time was identified using an ACT test, then the mixing study is performed using an ACT test. If the clotting time corrects after mixing, this likely represents a clotting factor deficiency. If the clotting time does not correct after mixing, this likely represents a clotting inhibitor (e.g., lupus anticoagulant).

In one example, a modified mixing study on a microfluidics device may include: (i) combining a patient plasma droplet with a normal pooled plasma droplet (e.g., at different ratios) to yield a combined test droplet; (ii) an activation step wherein the test droplet is combined with an activator reagent droplet for the desired clotting test; (ii) an initiation step to start the coagulation event (i.e., clotting); and (iii) a detection step to determine the time-to-clot formation (i.e., speed of clot formation).

In various embodiments, a combination of coagulation assays may be performed on a microfluidics device to assess deficiencies in clot formation. For example, a microfluidics device may be configured for performing two or more different coagulation tests in a multiplexed assay. In one example, a multiplexed assay may be used determine where a deficiency is in the coagulation cascade. In one example, a microfluidics device may be configured for performing modified aPTT, PT, and ACT assays described herein.

In various aspects, a method is provided for measuring blood coagulation that includes: (a) on a microfluidics device, merging a blood sample droplet with an activation reagent that includes a coagulation-activator and a coagulation initiation reagent to create a reaction droplet; (b) performing one or more shuttling impedance protocol cycles on the reaction droplet, wherein a single cycle comprises: transporting the reaction droplet from a first electrode across one or more electrodes to a destination electrode, wherein a measurement of impedance is taken at one or both the first and the destination electrodes; and (c) determining an ability of the blood sample droplet to form a clot and/or a time to clot formation when a change in the measured impedance meets or surpasses a predetermined threshold indicative of clot formation. Optionally, the measurement of impedance can be taken early enough in a settling time of the droplet for the measurement to be representative of droplet mobility, e.g., within 100 ms or less or within about 50 ms or less.

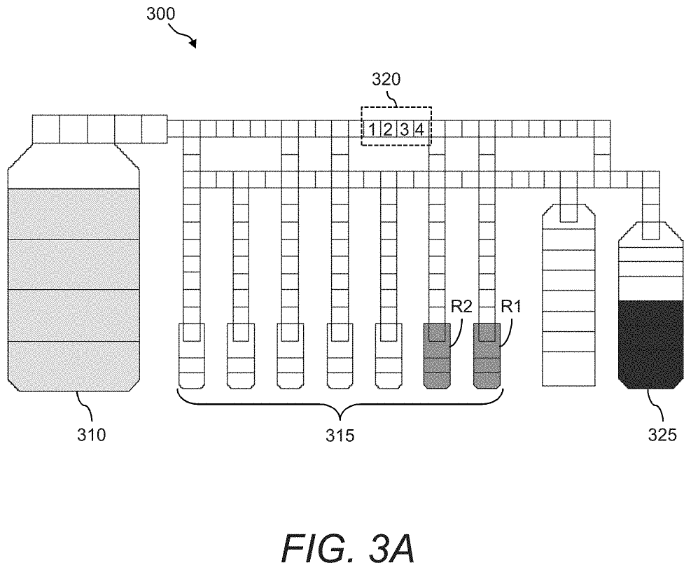

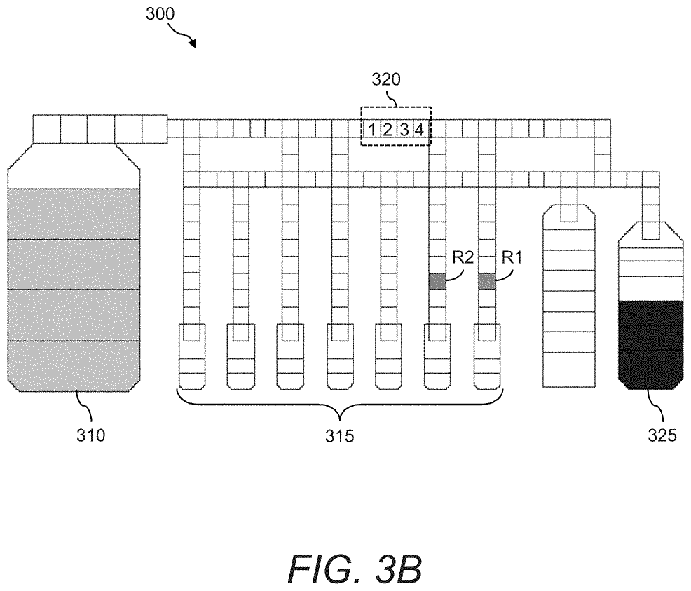

A single cycle of the shuttling impedance protocol cycles may include transporting the reaction droplet from the first electrode to the destination electrode and transporting the reaction droplet back to the first electrode which becomes the destination electrode as described below with reference to FIG. 3A and FIG. 3B. In other cases, the droplet may be transported on a long path (for example, a loop), with impedance measured at one or both of each new destination/trailing electrode.

Referring now to FIG. 3A and FIG. 3B is schematic diagrams of an example of an electrode arrangement 300. In electrode arrangement 300, droplet operations electrodes may be configured for conducting an aPTT assay for blood clotting on a microfluidics device. Electrode arrangement 300 may include a diluent reservoir 310 for dispensing a diluent solution. Further, electrode arrangement 300 may include one or more reagent reservoirs 315 for holding liquid reagents. For example, reagent reservoirs 315 may be used for dispensing the one or more liquid assay reagents (e.g., a first assay reagent R1 and a second assay reagent R2). Further, electrode arrangement 300 may include an assay reaction zone 320 for performing a shuttling impedance protocol. Further, electrode arrangement 300 may include a sample reservoir 325 for loading and dispensing a blood sample.

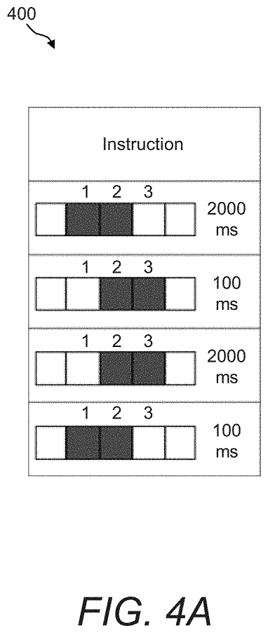

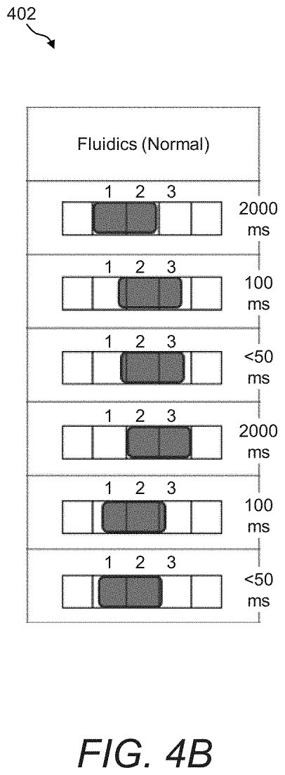

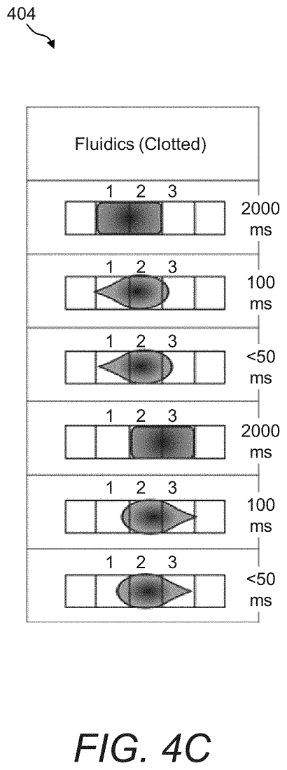

In this example, reaction zone 320 may include four droplet operations electrodes 1, 2, 3, and 4. The droplet operations electrodes 1, 2, 3, and 4 may be used for performing a shuttling impedance protocol on a sample reaction droplet (not shown) that is three (3) droplet units (DU) in size. Droplet operations electrodes 1 and 4 may be designated as “destination” electrodes where impedance measurements may be made. For example, a 3DU sample reaction droplet may be repeatedly shuttled back and forth across the four (4) droplets operations electrodes for a defined period (e.g., every two (2) seconds), with impedance at destination electrodes 1 and 4 checked at a certain point (e.g., <100 ms) into each shuttling cycle. An example of a shuttling impedance protocol is described in more detail below with reference to FIG. 4A, FIG. 4B, and FIG. 4C.

In some embodiments, one (1) or more assay reagents may be provided as dried reagent spots on certain droplet operations electrodes of electrode arrangement 300. For example, a first assay reagent R1 (e.g., an activation reagent) may be provided as a dried reagent spot on a droplet operations electrode arranged in a line or path with a first reagent reservoir 315. Likewise, a second assay reagent R2 (e.g., an initiation reagent) may be provided as a dried reagent spot on a droplet operations electrode arranged in a line or path with a second reagent reservoir 315. FIG. 3B shows an example of the first dried assay reagent R1 and the second dried assay reagent R2 at respective droplet operations electrodes. The dried reagents R1 and R2 are then rehydrated using, for example, the diluent solution provided in diluent reservoir 310 prior to conducting the assay.

For example, referring now to reaction zone 320 in electrode arrangement 300 shown in FIG. 3A and FIG. 3B, the shuttling impedance protocol may include, but is not limited to, the steps of:
(1) holding the 3DU reaction droplet across electrodes 1-3 for two (2) seconds of stay time;
(2) reading the impedance at electrodes 1 and 4, labeled with the replicate number (1-4), electrode (left, right), droplet position (left), and read type (stay);
(3) moving the 3DU reaction droplet to electrodes 2-4 for 100 ms of settling time;
(4) read the impedance at electrodes 1 and 4, labeled with the replicate number (1-4), electrode (left, right), droplet position (right), and read type (move);
(5) holding the 3DU reaction droplet across electrodes 2-4 for two (2) seconds of stay time;
(6) reading the impedance at electrodes 1 and 4, labeled with the replicate number (1-4), electrode (left, right), droplet position (right), and read type (stay);
(7) moving the 3DU reaction droplet to electrodes for 100 ms of settling time; and
(8) reading the impedance at electrodes 1 and 4, labeled with the replicate number (1-4), electrode (left, right), droplet position (left), and read type (move).

The methods of the disclosure use impedance measurements collected on the microfluidics device as the coagulation assay readout to determine the presence or absence of a clot and/or the speed of clot formation in a blood sample reaction droplet. Impedance readings may be collected at certain “destination” droplet operations electrodes positioned to detect how quickly a reaction droplet moves and to provide a measure of the amount of sample reaction fluid that is present at the electrode, i.e., how much current is measured at a destination electrode for a certain applied voltage is related to how much reaction fluid is present at the destination electrode. The impedance reads may be used as a surrogate measure to quantify droplet mobility. For example, droplet presence at a destination electrode at a certain time point indicates a droplet moving relatively quickly and droplet absence at a destination electrode at that time point indicates a droplet moving relatively slowly.

In some embodiments, the impedance measurements are used in a velocity-based detection process, wherein the rate of change in the position of a blood sample reaction droplet with respect to a frame of reference is determined as a function of time. For example, a reaction droplet's motion may be relatively fluid at first and may then quickly slow down becoming relatively “sluggish” as a clot forms.

Referring now to FIG. 4A, FIG. 4B, and FIG. 4C is schematic diagrams of examples of droplet shuttling impedance protocols 400, 402, and 404, respectively, for measuring time to clot formation. In these protocols, a reaction zone includes three (3) droplet operations electrodes (e.g., 1, 2, and 3) for performing a shuttling impedance protocol on a 2DU blood sample droplet.

Droplet shuttling impedance protocol 400 of FIG. 4A shows electrode activation/deactivation instructions for droplet shuttling across the three droplet operations electrodes 1, 2, and 3 in the reaction zone. Here, droplet operations electrodes 1 and 3 are designated as destination electrodes, and gray coloring indicates electrodes that are active (ON).

For example, droplet operations electrodes 1 and 2 are activated (ON) for 2 seconds (i.e., 2000 ms) of droplet hold or “stay” time. Then, droplet operations electrode 3 is activated (ON) and electrode 1 is deactivated (OFF) to move a sample droplet to electrodes 2 and 3 for 100 ms of “settling” time and impedance is measured at electrodes 1 and 3. Then, droplet operations electrodes 2 and 3 are held ON for two (2) seconds of stay time. Then, droplet operations electrode 1 is activated (ON) and electrode 3 is deactivated (OFF) to move the sample droplet to electrodes 1 and 2 for 100 ms of settling time and impedance is measured at electrodes 1 and 3. In other embodiments, impedance is measured at one of the two destination electrodes only.

Droplet shuttling impedance protocol 402 of FIG. 4B shows the fluidic movement of a normal sample droplet (i.e., not clotted) using the droplet shuttling impedance protocol described with reference to droplet shuttling impedance protocol 400. A normal sample reaction droplet will cover or substantially cover the destination electrode (i.e., impedance-check electrode) relatively quickly (e.g., by the 100 ms mark).

Droplet shuttling impedance protocol 404 of FIG. 4C shows the fluidic movement of a clotted sample reaction droplet using the droplet shuttling impedance protocol described with reference to droplet shuttling impedance protocol 400. A clotted sample reaction droplet will not complete the movement quickly enough (i.e., the reaction droplet moves relatively slowly) to cover or substantially cover the destination electrode (i.e., the impedance-check electrode) for the impedance detection.

In some embodiments, the time to formation of a clot in a blood sample reaction droplet may be used as an indicator of the severity of clot formation. For example, a sudden change in droplet mobility identified by a spike in impedance may indicate “severe” clot formation, while a more gradual change in droplet mobility identified by a relatively gradual increase in impedance may indicate less severe clotting.

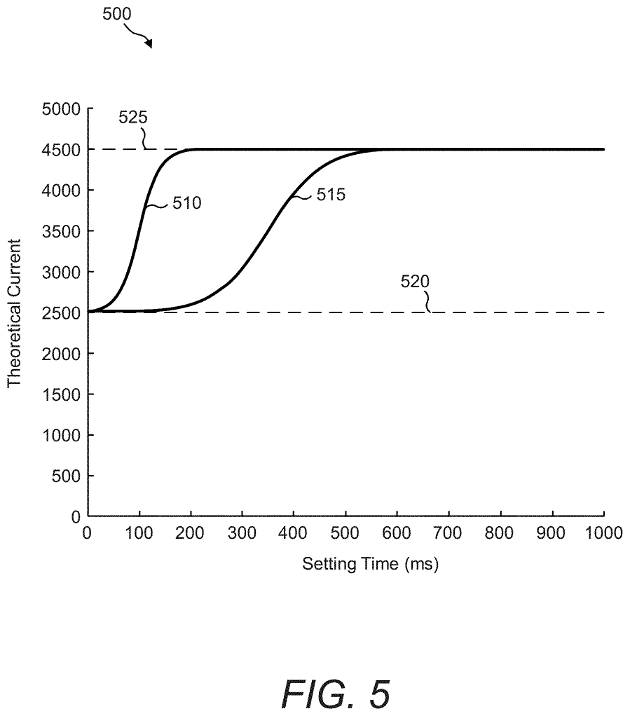

Referring now to FIG. 5 is a plot 500 showing an example of detecting droplet mobility using impedance measurements. Settling time in (seconds) is plotted along the X-axis (0-1,000) and theoretical current is plotted along the Y-axis (0-5,000). In this example, it is assumed that impedance can be measured constantly without affecting droplet movement. Plot 500 shows a plot line 510 of the theoretical current for a normal droplet (i.e., not clotted) and a plot line 515 of the theoretical current for a clotted droplet measured at a destination electrode over a period of one (1) second.

Plot 500 also shows an oil reference line 520 of theoretical impedance measured for an oil filler fluid (i.e., the oil filler fluid in the microfluidics device) and an aqueous droplet reference line 525. In this example, if a droplet moves quickly, i.e., a normal droplet depicted in plot line 510, then the impedance transitions relatively quickly from the oil impedance level (plot line 520) to the aqueous droplet reference level (plot line 525). If a droplet moves more slowly, i.e., a clotted droplet depicted in plot line 515, then the impedance transition from the oil impedance level (plot line 520) to the aqueous droplet reference level (plot line 525) is slower. By measuring impedance at least once early in a settling time, a sudden change in droplet mobility that accompanies clot formation may be monitored.

In some embodiments, the methods of the disclosure use a microfluidics device that includes an arrangement of droplet operations electrodes that are configured for evaluating coagulation of a blood sample, as described above with reference to FIG. 2, FIG. 3A, and FIG. 3B. All assay reagents including, for example, diluent, activation reagent, and initiation reagent for conducting the coagulation assay may be provided “pre-loaded” on the microfluidics device. In one example, the diluent is provided as a liquid reagent. Additionally, the activation and initiation reagents are provided as dried reagents that are rehydrated using the diluent solution. For example, rehydrate prior to dispensing a sample droplet and conducting the assay. Examples of reagents include, but are not limited to, diluent (e.g., 0.1% Tween 20, 0.05% Methylparaben), activation reagent (e.g., ellagic acid activator+phospholipids), and initiation reagent (e.g., CaCl₂). In other instances, the activation reagent also includes 20 mg/mL sucrose or 5 mg/mL trehalose, and the initiation reagent includes 1 mg/mL BSA or 5 mg/mL BSA.

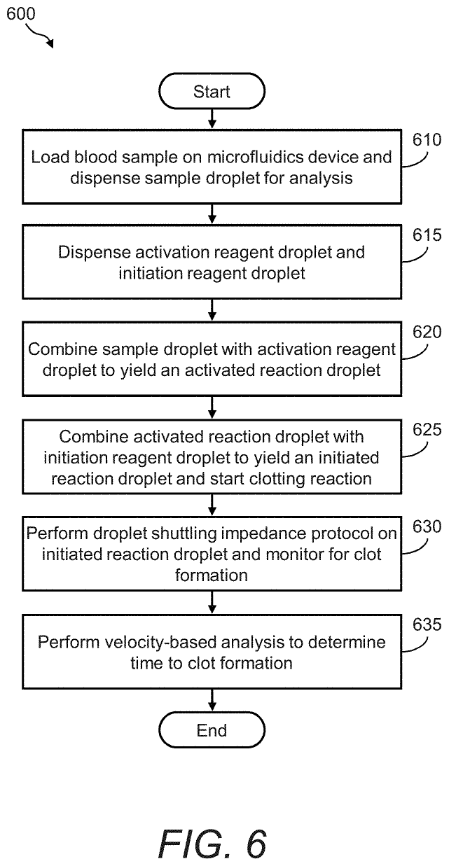

Referring now to FIG. 6 is a flow diagram of an example of a method 600 of measuring clotting of a blood sample on a microfluidics device using an aPTT assay. Method 600 may include, but is not limited to, the following steps as well as additional unspecified steps.

At a step 610, a blood sample is loaded onto a microfluidics device and a sample droplet is dispensed for analysis. For example, a blood sample is loaded into a sample reservoir of the microfluidics device and a 1DU sample droplet is dispensed. In one example, the blood sample is a plasma sample.

At a step 615, an activation reagent droplet and an initiation reagent droplet are dispensed. For example, a 1DU activation reagent droplet and a 1DU initiation reagent droplet are dispensed.

At a step 620, the sample droplet is combined with the activation reagent droplet to yield an activated reaction droplet. For example, the 1DU sample droplet is combined with the 1DU activation reagent droplet to yield a 2DU activated reaction droplet and incubated for a period of time (e.g., about three (3) minutes) sufficient for activation of the clotting process.

At a step 625, the activated reaction droplet is combined with the initiation reagent droplet to start the clotting reaction. For example, the 2DU activated reaction droplet is combined with the 1DU initiation reagent droplet to yield a 3DU reaction droplet. The 3DU reaction droplet is mixed for about fifteen (15) seconds or less to start the clotting process.

At a step 630, a droplet shuttling impedance protocol is performed on the initiated reaction droplet and monitored for clot formation. In one example, the droplet shuttling impedance protocol includes forty (40) replicates of the shuttling cycle.

At a step 635, a velocity-based analysis is performed to determine the time to clot formation. It is noted that in this example of the method and velocity-based analysis, the electric current is determined from the impedance measurements. A measurement of impedance is detected, and a measurement of current output is used for analysis.

For example, using a parenthetical pair, such as (L,R), as shorthand for “droplet going Left, impedance of Right electrode”, an analysis method may include:
(1) consider only the magnitude value of the impedance results outputted as current:

for all four (4) pairs (L,R), (L,L), (R,L), and (R,R), subtract their first reading from all of their forty (40) readings each (i.e., baseline them to their initial value);
(2) average (L,R) and −(R,R) together to for (x,R), which is the “change in droplet current when droplet moves right”; and average (R,L) and −(L,L) together to form (x,L), which is the “change in current when the droplet moves left”;
(3) combine (x,R) and (x,L) in a zipper fashion (i.e., first of one, first of the other, second of one, second of the other, etc.) forming the raw combined signal Y; combine their timestamps in the same way forming the Time vector;
(4) smooth signal [Time, Y] with a moving average filter (span of five (5)) to produce smoothed signal Y_s; and linearly interpolate [Time, Y_s] to find when Y_s is first>Z impedance units outputted as current, where Z=200; the corresponding time is the clot time. In some cases, Z can also=100, giving a slightly reduced clot time and greater sensitivity to the assay.

### Applications

The system, microfluidics device, and methods of the disclosure may be used for monitoring the status of a therapy or selecting a drug and/or drug dosage for a therapy.

In one embodiment, the system, microfluidics device, and methods may be used for monitoring the status of an anticoagulant therapy. In one example, the method may include, but is not limited to, the steps of:
(1) providing a microfluidics device that includes:
   a. an arrangement of droplet operations electrodes configured for evaluating coagulation of a blood sample;
   b. an activation reagent; and
   c. an initiating reagent (e.g., CaCl₂)
   wherein the microfluidics device and reagents are configured for performing a selected coagulation assay;
(2) providing a patient blood sample (e.g., a plasma sample);
(3) dispensing a blood sample droplet;
(4) combining the sample droplet with an activation reagent to yield a test droplet;
(5) combining the test droplet with an initiating reagent to yield an initiated test droplet;

(6) performing a droplet shuttling impedance protocol on the initiated test droplet to determine clot formation and/or time-to-clot formation; and (7) evaluating the impedance data to determine whether an adjustment to the therapy is required.

In one embodiment, the system, microfluidics device, and methods may be used for selecting a drug dosage for an anticoagulant therapy. In one example, the method may include, but is not limited to, the steps of:
(1) providing a microfluidics device that includes:
   a. an arrangement of droplet operations electrodes configured for evaluating coagulation of a blood sample;
   b. an activation reagent;
   c. an initiating reagent (e.g., CaCl₂); and
   d. a range of anticoagulant drug concentrations;
   wherein the microfluidics device and reagents are configured for selecting a drug dosage for an anticoagulant therapy;
(2) providing a patient blood sample (e.g., a plasma sample);
(3) dispensing a blood sample droplet;
(4) combining the blood sample droplet with an anticoagulant at a test concentration to yield a test droplet;
(5) combining the test droplet with an activation reagent to yield an activated test droplet;
(6) combining the activated test droplet with an initiating reagent to yield an initiated test droplet;
(7) performing a droplet shuttling impedance protocol on the initiated test droplet to monitor clot formation;
(8) repeating steps (3) through (7) for each anticoagulant drug concentration; and
(9) evaluating clot formation across the range of anticoagulant concentrations to determine an effective concentration of anticoagulant to use for therapy.

The system, microfluidics device, and methods of the disclosure may be used for analyzing the addition and/or depletion of clotting factors in coagulation of a blood sample to assess clotting efficiency in a blood sample. For example, a clotting factor may be depleted from a blood sample to assess the effects on clotting, and a clotting factor may be added to the blood sample to assess the effects on clotting, and the differences in clot formation and/or time-to-clot formation may be compared and evaluated.

### EXAMPLES

Generally, in the blood coagulation assays of the disclosure, one or more observation methods may be used to determine how the speed of a droplet changes—either the leading edge or trailing edge or both. From this, the clotting time and/or clottability of the droplet may be deduced or inferred. Generally, the disclosure provides systems, devices, and methods to measure the change in viscoelastic properties of a blood droplet.

The method of the disclosure uses impedance measurements collected on the microfluidics device as the coagulation assay readout to determine the presence or absence of a clot and/or the speed of clot formation in a blood sample reaction droplet.

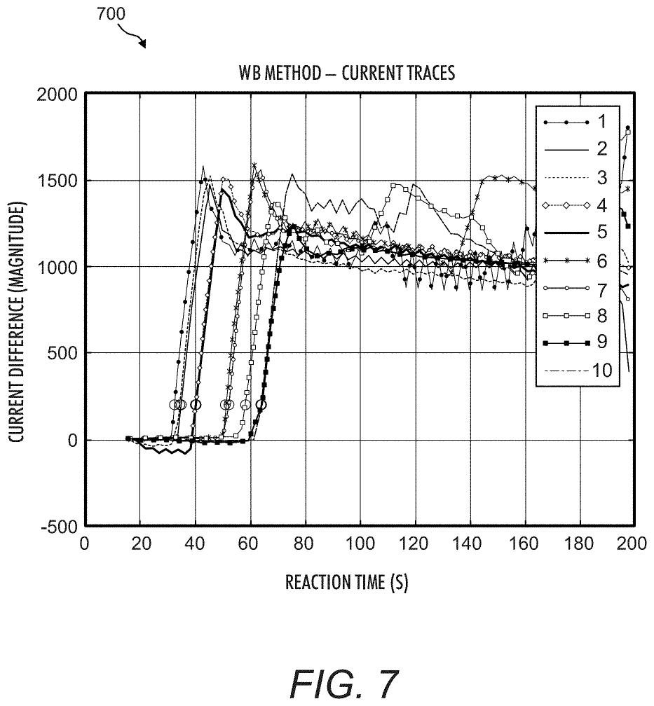

Referring now to FIG. 7 is a plot 700 showing impedance traces for clot formation in whole blood (WB) reaction droplets. In this example, reaction time in (seconds) (0-200) is plotted along the X-axis and current difference (magnitude) is plotted along the Y-axis (−500 to 2,000). The plot lines represent the traces of ten different samples at a single shuttling protocol. The data show that once a clot forms, there is a measurable change in the magnitude of current. This change in current may be readily characterized using an automated algorithm.

To determine whether the impedance reads are measuring droplet mobility and not conflated with changes in other electrical characteristics, the droplet shuttling impedance protocol was executed on six (6) different instruments using one (1) blood sample and a variety of settling times (i.e., the time the droplet gets to move before impedance is measured).

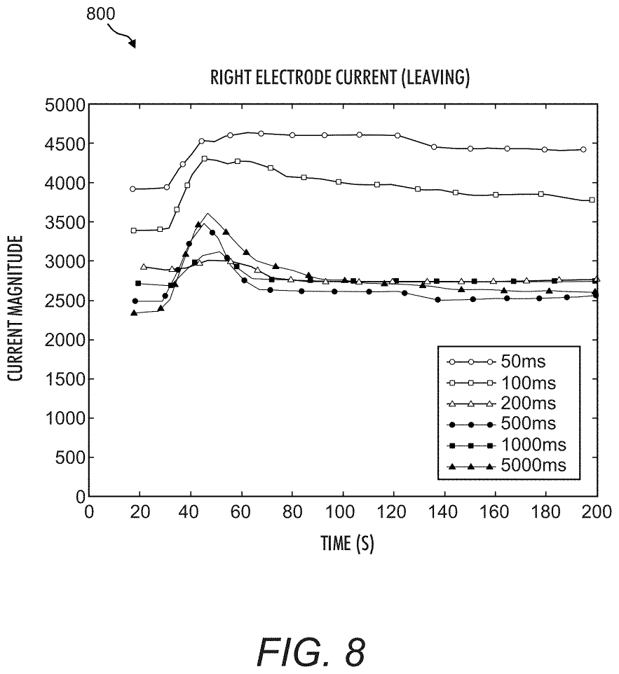

Referring now to FIG. 8 is a plot 800 showing impedance traces for clot formation in a blood sample at different settling times in a droplet shuttling impedance protocol. Time in (seconds) is plotted along the X-axis (0-200) and current magnitude is plotted along the Y-axis (0-5,000). The plot lines are 50 ms, 100 ms, 200 ms, 500 ms, 1,000 ms, and 2,000 ms. In this example, current measurements are shown for the electrode the reaction droplets are leaving (i.e., the Right destination electrode) during the droplet shuttling impedance protocol. The data show that the “bump” in current magnitude that indicates clot formation is diminished by the increased settling time, indicating that other changes in electrical characteristics are not masking current measurements.

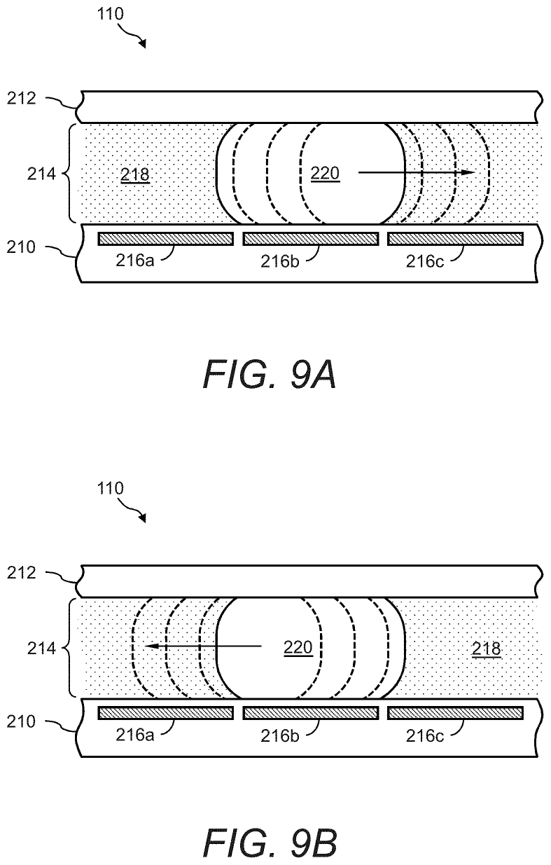

Referring now to FIG. 9A and FIG. 9B is cross-sectional views of a portion of microfluidics device 110 and showing an example of a droplet shuttling impedance protocol for use in the blood coagulation assays of the disclosure. In this example, microfluidics device 110 may include droplet operations electrodes 216a, 216b, 216c. FIG. 9A shows a droplet 220 atop droplet operations electrodes 216b (i.e., a first electrode) and then being shuttled back and forth between droplet operations electrodes 216b and 216c. Droplet 220 being the droplet under test.

FIG. 9A shows shuttling impedance protocol cycles on droplet 220 comprising transporting droplet 220 from a starting electrode (e.g., droplet operations electrodes 216b) to a destination electrode (e.g., droplet operations electrodes 216c), wherein a measurement of impedance may be taken at one or both droplet operations electrodes 216b and 216c early enough in time for the measurement to be representative of droplet mobility.

For example, sensors (not shown) may be embedded at each droplet operations electrode 216 to measure impedance. These sensors may be driven and/or controlled by circuitry of impedance sensing 130. Accordingly, impedance sensing 130 may be used to monitor the impedance of droplet operations electrodes 216b and 216c during the shuttling impedance protocol. Accordingly, impedance sensing 130 may be used to determine a change in viscoelastic properties of droplet 220 by determining a change in the measured impedance. Impedance information is available at both the advancing and receding electrode. For example, impedance sensing 130 may be used to (1) measure how the leading edge of droplet 220 is moving onto the destination electrode (e.g., droplet operations electrodes 216c), and (2) measure how the trailing edge of droplet 220 is receding off the starting electrode (e.g., droplet operations electrodes 216b).

FIG. 9B shows substantially the same shuttling impedance protocol as shown in FIG. 9A, except that droplet operations electrodes 216b is the starting electrode and droplet operations electrodes 216a is the destination electrode.

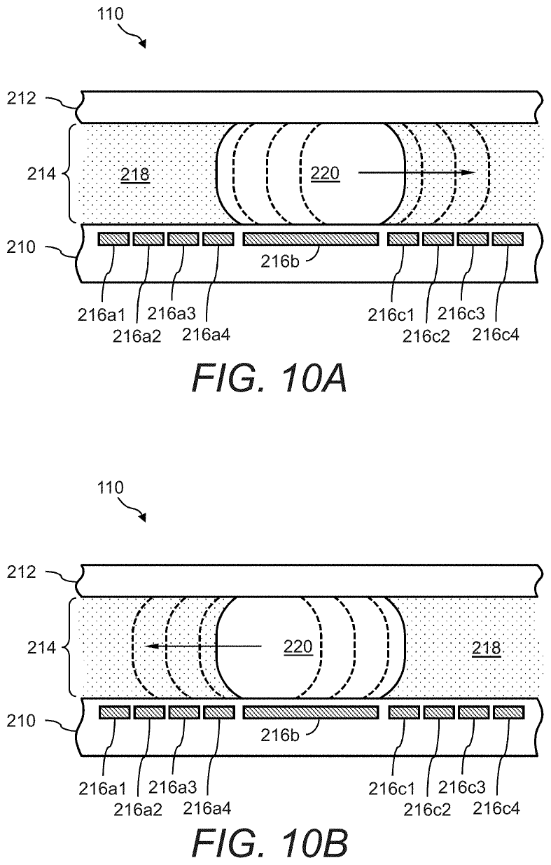

Referring now to FIG. 10A and FIG. 10B is cross-sectional views of a portion of microfluidics device 110 and showing another example of a droplet shuttling impedance protocol for use in the blood coagulation assays of the disclosure. This droplet shuttling impedance protocol is substantially the same as the droplet shuttling impedance protocol shown and described in FIG. 9A and FIG. 9B, except that the 1DU destination electrodes (droplet operations electrodes 216a, 216c) have been replaced with multiple smaller electrodes. For example, the 1DU droplet operations electrode 216a has been replaced with four (4) smaller or narrower droplet operations electrodes 216a1, 216a2, 216a3, 216a4. Likewise, the 1DU droplet operations electrode 216c has been replaced with four (4) smaller or narrower droplet operations electrodes 216c1, 216c2, 216c3, 216c4.

The smaller droplet operations electrodes 216a1, 216a2, 216a3, 216a4 and 216c1, 216c2, 216c3, 216c4 may be beneficial to provide a stronger cleaner signal for the impedance measurements. Accordingly, droplet operations electrodes 216a1, 216a2, 216a3, 216a4 and 216c1, 216c2, 216c3, 216c4 may be used to provide more precise impedance measurements and thereby provide more precise droplet position information as compared with the 1DU droplet operations electrodes 216. That is, improved “over time” and “over space” information.

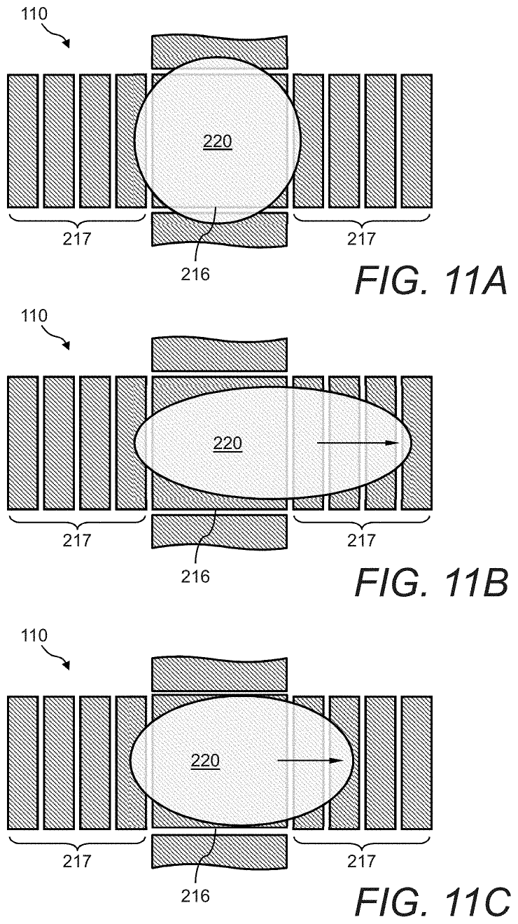

Referring now to FIG. 11A, FIG. 11B, and FIG. 11C is plan views showing an example of an electrode configuration including flanking electrodes and showing another example of a droplet shuttling impedance protocol for use in the blood coagulation assays of the disclosure. In this example, a 1DU droplet operations electrode 216 may be flanked on each side by an arrangement of smaller flanking electrodes 217. For example, four (4) flanking electrodes 217 may be about the same area as one 1DU droplet operations electrode 216. Again, impedance sensing 130 may be used to monitor the impedance of flanking electrodes 217 during the shuttling impedance protocol and determine the viscoelastic properties of droplet 220.

In the droplet shuttling impedance protocol shown in FIG. 11A, FIG. 11B, and FIG. 11C just one set of flanking electrodes 217 may be used. For example, FIG. 11A shows droplet 220 atop droplet operations electrode 216 and with flanking electrodes 217 not activated (OFF). Droplet 220 being the droplet under test. Then, early in time in the protocol and wherein droplet 220 may be highly fluid, FIG. 11B shows one set of flanking electrodes 217 activated (ON) and droplet 220 spreading across several flanking electrodes 217. Then, later in time in the protocol and wherein droplet 220 may be less fluid as coagulation occurs, FIG. 11C shows one set of flanking electrodes 217 activated (ON) and droplet 220 now spreading across fewer flanking electrodes 217.

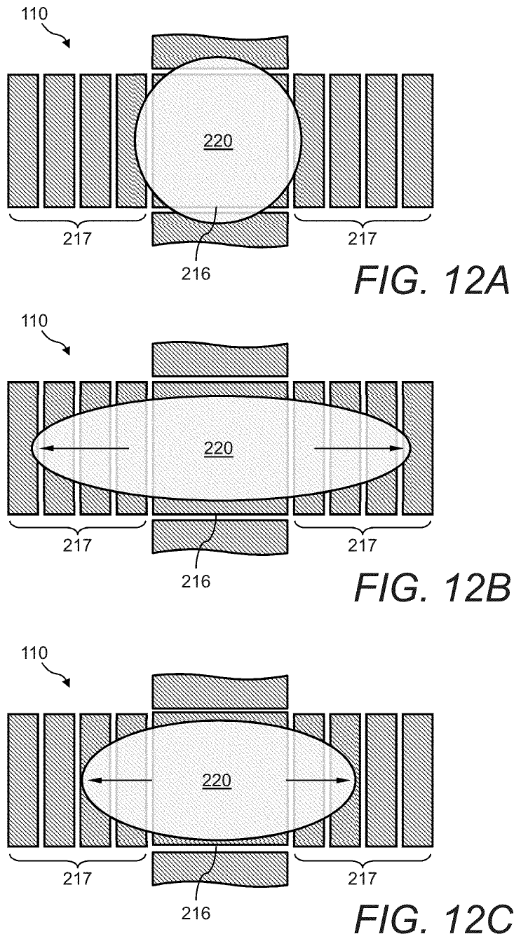

Referring now to FIG. 12A, FIG. 12B, and FIG. 12C is plan views showing the electrode configuration shown in FIG. 11A, FIG. 11B, and FIG. 11C and showing another example of a droplet shuttling impedance protocol for use in the blood coagulation assays of the disclosure. The droplet shuttling impedance protocol shown in FIG. 12A, FIG. 12B, and FIG. 12C is substantially the same as the droplet shuttling impedance protocol shown in FIG. 11A, FIG. 11B, and FIG. 11C, except that both sets of flanking electrodes 217 are being used instead of just one set. Again, impedance sensing 130 may be used to monitor the impedance of flanking electrodes 217 during the shuttling impedance protocol and determine the viscoelastic properties of the droplet 220 of interest.

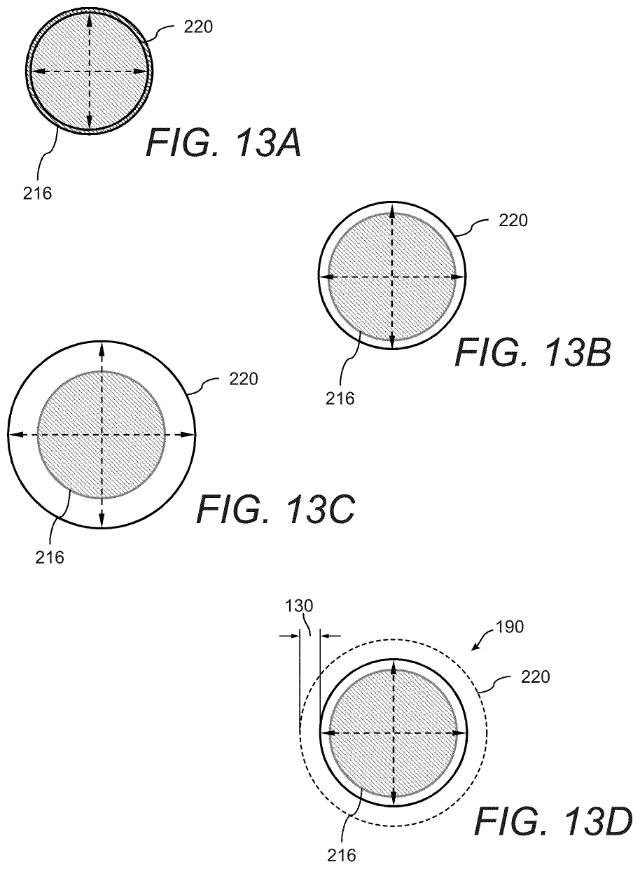

Referring now to FIG. 13A, FIG. 13B, FIG. 13C, and FIG. 13D is plan views showing an example of a circular electrode configuration 190 and showing another example of a droplet shuttling impedance protocol for use in the blood coagulation assays of the disclosure. In this example, during the droplet shuttling impedance protocol, the droplet of interest may be caused to expand and contract atop circular electrode configuration 190. At the same, impedance sensing 130 may be used to monitor how the movement of the droplet 220 changes over time.

For example, FIG. 13A shows that circular electrode configuration 190 may include a circular droplet operations electrode 216 arranged within a ring-shaped droplet operations electrode 216. Circular electrode configuration 190 is not limited to one ring-shaped droplet operations electrode 216 only. In other examples, circular electrode configuration 190 may include multiple larger and larger ring-shaped droplet operations electrodes 216 arranged concentrically around the circular droplet operations electrode 216.

Next, FIG. 13B shows a droplet 220 sitting atop the circular droplet operations electrode 216. For example, the circular droplet operations electrode 216 may be activated (ON) while the ring-shaped droplet operations electrode 216 may be deactivated (OFF). Accordingly, droplet 220 may sit substantially atop the circular droplet operations electrode 216 only. Here, droplet 220 may be considered in the “contracted” state.

Next, FIG. 13C shows both the circular droplet operations electrode 216 and the ring-shaped droplet operations electrode 216 may be activated (ON). This may cause droplet 220 to expand outward covering both the circular droplet operations electrode 216 and the ring-shaped droplet operations electrode 216. Here, droplet 220 may be considered in the “expanded” state.

Next, FIG. 13D shows the circular droplet operations electrode 216 activated (ON) while the ring-shaped droplet operations electrode 216 may be deactivated (OFF). This allows droplet 220 to return to the “contracted” state atop circular droplet operations electrode 216.

Accordingly, the droplet shuttling impedance protocol may include activating (ON) circular droplet operations electrode 216 and then cycling the ring-shaped droplet operations electrode 216 ON and OFF. This may cause droplet 220 to cycle between the “contracted” state and the “expanded” state. All the while, impedance sensing 130 may be used to monitor how the movement of the droplet 220 changes over time.

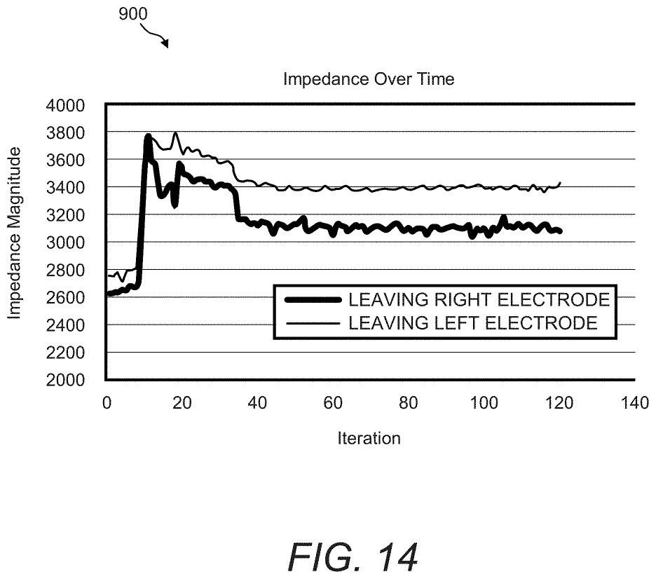

Referring now to FIG. 14 illustrates a plot 900 showing impedance traces for example clot formation of a droplet shuttling impedance protocol. Plot 900 may be representative of the impedance measurements taken during any of the droplet shuttling impedance protocols described in FIG. 9A through FIG. 13D and suitable for use in the blood coagulation assays of the disclosure. Plot 900 shows that the droplet starts by moving rapidly. Then the droplet movement slows as the viscosity increases.

Generally, in the droplet shuttling impedance protocols described in FIG. 9A through FIG. 13D, the impedance measurement information may be processed and correlated to the viscoelastic properties of the droplet 220 of interest.

Further, in the droplet shuttling impedance protocols described in FIG. 9A through FIG. 13D, the impedance measurement may be taken just once in time after imitating the droplet movement. For example, the impedance measurement may be taken about 50 ms after imitating the droplet movement. In this example, if the droplet has moved quickly (a highly fluid droplet), then a significant change in impedance may be detected. However, if the droplet has moved slowly (a less fluid droplet), then little or no change in impedance may be detected. However, in another example, the impedance may be sampled continuously in such a way as to provide a precise plot of the droplet movement.

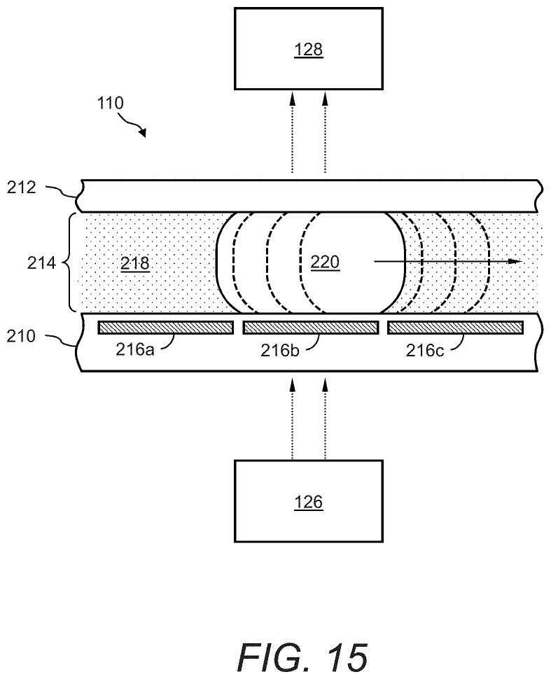

Referring now to FIG. 15 is a cross-sectional view of a portion of microfluidics device 110 and showing an example of a droplet shuttling optical imaging protocol for use in the blood coagulation assays of the disclosure. In this example, the droplet shuttling optical imaging protocol may use detection system 124 to determine the viscoelastic properties of the droplet 220 of interest. For example, illumination source 126 may be arranged on one side of microfluidics device 110 and optical measurement device 128 (e.g., color camera) may be arranged on the opposite side of microfluidics device 110.

Droplet shuttling optical imaging protocol may be substantially the same as the shuttling impedance protocol shown in FIG. 9A, except that digital imaging processes may be used instead of impedance measurements to determine the viscoelastic properties of the droplet 220 of interest. That is, during the droplet shuttling optical imaging protocol, optical measurement device 128 (e.g., color camera) may be used to provide “visual” observation of the position and/or movement of droplet 220. The image information is then processed and correlated to the viscoelastic properties of the droplet 220 of interest.

Referring still to FIG. 15, a droplet shuttling optical imaging protocol for measuring viscoelastic properties in a droplet may include: (a) dispensing a sample droplet 220 on microfluidics device 110 and, optionally, merging the droplet 220 with one or more reaction reagents; (b) on the microfluidics device 110, performing one or more shuttling optical imaging protocol cycles on the droplet 220 comprising transporting the droplet 220 from a first electrode (e.g., 216b) to a destination electrode (e.g., 216c), wherein a digital image is captured at one or both electrodes using optical measurement device 128 (e.g., color camera); and (c) using a computer (e.g., controller 114) with image processing capabilities, determining a change in viscoelastic properties of the droplet 220 to determine, for example, a change in the size, shape, color, and/or position of the droplet 220. Optionally, the digital image may be captured early enough in a settling time of the droplet 220 for the contents of the digital image to be representative of droplet mobility, e.g., within 100 ms or less or within about 50 ms or less.

Further, and referring now to FIG. 1 through FIG. 15, in the droplet shuttling protocols described above, other variations are possible. For example, as the viscosity of a droplet increases, the electrowetting voltage must increase to make a droplet move. Therefore, for a given the electrowetting voltage, the protocol may determine how the droplet movement changes with any change in viscoelastic properties. Further, the droplet may even stop moving at certain viscosity. Therefore, the protocol may be used to monitor for the droplet “stop” voltage. Then, increase the electrowetting voltage some amount to see where the droplet moves again. For example, increase by +2v, then another +2v, and so on. For a certain viscosity, this may be called the “movement threshold voltage.”

In another example, the electrowetting voltage or force applied to move the droplet may vary throughout the droplet shuttling protocol. This may be useful for learning more about the viscoelastic properties of the droplet. In this example, the electrowetting voltage or force may be used as a tunable input of the protocol.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The terms “comprise,” “comprises,” “comprising,” “include,” “includes,” and “including,” are intended to be non-limiting, such that recitation of items in a list is not to the exclusion of other like items that may be substituted or added to the listed items.

Terms like “preferably,” “commonly,” and “typically” are not used herein to limit the scope of the claimed embodiments or to imply that certain features are critical or essential to the structure or function of the claimed embodiments. These terms are intended to highlight alternative or additional features that may or may not be used in a particular embodiment of the disclosure.

The term “substantially” is used herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

Various modifications and variations of the disclosed methods, compositions and uses of the disclosure will be apparent to the skilled person without departing from the scope and spirit of the disclosure. Although the subject matter has been disclosed in connection with specific preferred aspects or embodiments, it should be understood that the subject matter as claimed should not be unduly limited to such specific aspects or embodiments.

The subject matter may be implemented using hardware, software, or a combination thereof and may be implemented in one or more computer systems or other processing systems. In one aspect, the subject matter is directed toward one or more computer systems capable of carrying out the functionality described herein.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other factors known to those of skill in the art depending on the desired properties sought to be obtained by the subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The subject matter may be embodied in many different forms and should not be construed as limited to the embodiments set forth herein; rather, these embodiments are provided so that this disclosure will satisfy applicable legal requirements. Many modifications and other embodiments of the subject matter set forth herein will come to mind to one skilled in the art to which the subject matter pertains having the benefit of the teachings presented in the foregoing descriptions and the associated drawings. The subject matter is not to be limited to the specific embodiments disclosed and that modifications and other embodiments are intended to be included within the scope of the appended claims.

We claim:

1. A method for measuring blood coagulation, the method comprising:
a. on a microfluidics device, merging a blood sample droplet with an activation reagent comprising a coagulation-activator and with a coagulation initiation reagent to create a reaction droplet;
b. performing one or more shuttling impedance protocol cycles on the reaction droplet, wherein a single cycle of the one or more shuttling impedance protocol cycles comprises: transporting the reaction droplet from a first electrode across one or more electrodes to a destination electrode, wherein a measurement of impedance is taken at one or both of the first electrode and/or the destination electrode; and
c. determining an ability of the blood sample droplet to form a clot when a change in the measured impedance meets or surpasses a predetermined threshold indicative of clot formation.

2. The method of claim 1, wherein performing the single shuttling impedance protocol cycle comprises:
a. holding the reaction droplet across the first electrode designated 1 and two electrodes designated 2 and 3 for about two seconds or more, wherein the reaction droplet is three reaction droplet units in size (3DU);
b. transporting the 3DU reaction droplet to electrodes 2-4 and holding for about 100 ms or less of settling time, wherein electrode 4 is the destination electrode;
c. taking a measurement of impedance at the first electrode and the destination electrode;
d. holding the 3DU reaction droplet across electrodes 2-4 for about two seconds or more;
e. transporting the 3DU reaction droplet to electrodes 1-3 and holding for about 100 ms or less of settling time; and
f. taking a measurement of impedance at the first electrode and the destination electrode.

3. A method for measuring blood coagulation, comprising:
a. using electrowetting-mediated droplet operations on a microfluidics device:
i. dispensing a droplet of a blood sample in a droplet operations gap of the microfluidics device,
ii. initiating an activated partial thromboplastin time (aPTT) test by merging the sample droplet with an activation reagent droplet and an initiation reagent droplet to create a reaction droplet; and
iii. performing one or more shuttling impedance protocol cycles on the reaction droplet, wherein a single cycle of the one or more shuttling impedance protocol cycles comprises transporting the reaction droplet from a first electrode to a destination electrode, wherein a measurement of impedance is taken at one or both of the first electrode and/or the destination electrode; and
b. using a computer, determining an ability of the blood sample droplet to form a clot when a change in the measured impedance meets or surpasses a predetermined threshold indicative of clot formation.

4. The method of any one of the preceding claims, the method further comprising measuring the change in impedance over time and determining a time to clot formation when a change in the measured impedance meets or surpasses a predetermined threshold indicative of clot formation.

5. The method of any one of the preceding claims, wherein the activation reagent comprises ellagic acid in a phospholipid solution or silica in a phospholipid solution.

6. The method of any one of the preceding claims, wherein the initiation reagent overcomes the effect of an anticoagulant agent used during initial collection of the blood sample.

7. The method of any one of the preceding claims, wherein the initiation reagent comprises CaCl₂.

8. The method of any one of the preceding claims, wherein the reaction droplet is held for a defined period of time after each measurement of impedance.

9. The method of claim 8, wherein the defined period of time comprises about 2 seconds or more.

10. The method of any one of the preceding claims, wherein the single cycle comprises transporting the reaction droplet from the first electrode across the one or more electrodes to the destination electrode and transporting the reaction droplet back to the first electrode which becomes the destination electrode.

11. The method of claim 11, wherein forty or fewer shuttling impedance protocol cycles are performed.

12. The method of any one of the preceding claims, wherein if the change in measured impedance is sudden it is indicative of more severe clot clotting, and wherein if the change in measured impedance is gradual it is indicative of less severe clotting.

13. The method of any one of the preceding claims, wherein the measurement of impedance is a measurement of current at the electrode for an applied voltage.

14. A method for measuring viscoelastic properties in a droplet, the method comprising:
a. dispensing a droplet of a sample on a microfluidics device and, optionally, merging the droplet with one or more reaction reagents;
b. on the microfluidics device, performing one or more shuttling impedance protocol cycles on the droplet, wherein the one or more shuttling impedance protocol cycles comprises transporting the droplet from a first electrode to a destination electrode, wherein a measurement of impedance is taken at one or both of the electrodes; and
c. using a computer, determining a change in viscoelastic properties of the droplet by determining a change in the measured impedance.

15. The method of claim 14, wherein the reaction droplet is transported from the first electrode across one or more electrodes to the destination electrode.

16. The method of any one of claims 14-15, wherein the one or more shuttling impedance protocol cycles comprises transporting the reaction droplet from the first electrode to the destination electrode and transporting the reaction droplet back to the first electrode which becomes the destination electrode.

17. The method of any one of claims 14-16, wherein the measurement of impedance is a measurement of current at the electrode for an applied voltage.

18. The method of any one of claims 14-17, wherein the measured current is indicative of an amount of the reaction droplet present at the electrode.

19. The method of any one of the preceding claims, wherein the measurement of impedance is taken early enough in a settling time of the reaction droplet for the measurement to be representative of droplet mobility, and wherein early enough in the settling time comprises about 100 ms or less.

20. The method of any one of the preceding claims, wherein the measurement of impedance is taken at each of the first and the destination electrodes.

21. The method of any one of the preceding claims, wherein the measurement of impedance is taken each time the reaction droplet is transported.

22. The method of any one of the preceding claims, wherein the sample droplet is a blood sample droplet comprising a test compound, wherein an effect of the test compound on blood coagulation is determined by the change in measured impedance relative to a reference blood sample droplet that does not comprise the test compound.

23. The method of any one of preceding claims, further comprising merging the blood sample droplet with a test compound, wherein an effect of the test compound on blood coagulation is determined by the change in measured impedance relative to a reference blood sample droplet that does not comprise the test compound.

24. The method of any one of the preceding claims, wherein the microfluidics device comprises one or more of a sample reservoir for loading and dispensing the sample, a diluent reservoir for dispensing a diluent solution, and one or more reagent reservoirs for dispensing one or more liquid assay reagents.

25. The method of any one of the preceding claims, wherein one or more of the reaction reagents is provided as a dried reagent spot on one or more droplet operations electrodes on the microfluidics device.

26. The method of claim 25, wherein the one or more dried reagent spots are rehydrated using a diluent solution provided in a diluent reservoir.

27. The method of any one of the preceding claims, wherein the microfluidics device comprises an electrowetting cartridge and the dispensing, merging, transporting, combining, and/or initiating is performed using electrowetting-mediated droplet operations.

28. The method of any one of the preceding claims, wherein the shuttling impedance protocol cycles are performed in a reaction zone on the microfluidics device.

29. The method of any one of the preceding claims, wherein the blood sample or the sample is a whole blood sample or a plasma sample.

30. The method of claim 29, wherein the plasma sample is prepared on the microfluidics device from a whole blood sample by combining a whole blood sample droplet with an agglutination reagent and separating a plasma fraction from a red blood cell fraction using a plasma separation process.

31. A system comprising a computer processor and an electrowetting cartridge wherein the processor is programmed to execute the method of any one of the preceding claims.

32. A kit comprising an electrowetting cartridge and reagents sufficient to execute the method of any one of the preceding claims.
